SEQUENCE LISTING (12 sequences)

SEQ ID NO: 1 - DNA, 22 bp, Homo sapiens
tggcctggagcaacaaatctga

SEQ ID NO: 2 - DNA, 22 bp, Homo sapiens
accggacctcgttgtttagact

SEQ ID NO: 3 - protein, 4 aa, Artificial Sequence
  Synthesized
QEED

SEQ ID NO: 4 - protein, 4 aa, Artificial Sequence
  Synthesized
EEEE

SEQ ID NO: 5 - protein, 4 aa, Artificial Sequence
  Synthesized
SEEE

SEQ ID NO: 6 - protein, 42 aa, Artificial Sequence
  Synthesized
KASRKKAAAAAKSPFASPASSAQEEDASSCRAPSEEEGSCEL

SEQ ID NO: 7 - protein, 42 aa, Artificial Sequence
  Synthesized
KRGRKKLLYIFKQPFMRPVQTTQEEDGCSCRFPEEEEGGCEL

SEQ ID NO: 8 - protein, 112 aa, Artificial Sequence
  Synthesized
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 10 - protein, 24 aa, Artificial Sequence
  Synthesized
IYIWAPLAGTCGVLLLSLVITLYC

SEQ ID NO: 11 - protein, 45 aa, Artificial Sequence
  Synthesized
TTTPAPRPPTPAPTIASQPLSLRPEACRPAAGGAVHTRGLDFACD

SEQ ID NO: 12 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

(19) United States
(12) Patent Application Publication
(10) Pub. No.: US 2022/0273720 A1
(43) Pub. Date: Sep. 1, 2022

(54) LYMPHODEPLETION DOSING REGIMENS FOR CELLULAR IMMUNOTHERAPIES

(71) Applicant: Precision BioSciences, Inc., Durham, NC (US)

(72) Inventor: Bruce J. McCreedy, Jr., Raleigh, NC (US)

(73) Assignee: Precision BioSciences, Inc., Durham, NC (US)

(21) Appl. No.: 17/636,726

(22) PCT Filed: Aug. 20, 2020
(86) PCT No.: PCT/US2020/047202

§ 371 Date: Feb. 18, 2022

Related U.S. Application Data
(60) Provisional application No. 63/061,070, filed on Aug. 4, 2020.
(60) Provisional application No. 62/889,338, filed on Aug. 20, 2019.

(51) Int. Cl. A61K 35/17; A61K 39/395; A61K 31/7076; A61K 31/675; A61K 31/4184; A61K 31/198; A61K 31/52; A61K 31/704; A61K 31/7068; A61K 31/519; A61K 31/573; A61K 38/50; A61K 38/17; A61P 37/04

(52) U.S. Cl. CPC A61K 35/17; A61K 39/3955; A61K 31/7076; A61K 31/675; A61K 31/4184; A61K 31/198; A61K 31/52; A61K 31/704; A61K 31/7068; A61K 31/519; A61K 31/573; A61K 38/50; A61K 38/1774; A61P 37/04

(57) ABSTRACT

The present invention encompasses methods and compositions including genetically-modified cells expressing chimeric antigen receptors or exogenous T cell receptors, and pharmaceutical compositions thereof, for the treatment of cancer and other disorders and diseases. Further, provided herein are methods for depleting lymphocytes in a subject in need of treatment prior to, concomitant with, or following administration of the genetically-modified cells provided herein.

### FIELD OF THE INVENTION

The invention relates to the field of oncology, cancer immunotherapy, molecular biology and recombinant nucleic acid technology. In particular, the invention relates to genetically-modified cells comprising a chimeric antigen receptor or exogenous T cell receptor, and methods and compositions related thereto for the treatment of cancer and other disorders and diseases. The invention further relates to methods and compositions for depleting lymphocytes in a subject prior to, concomitant with, or following treatment with the genetically-modified cells provided herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Aug. 20, 2020, is named P109070043WO00-SEQ-EPG, and is 8 kilobytes in size.

### BACKGROUND OF THE INVENTION

T cell adoptive immunotherapy is a promising approach for cancer treatment. This strategy utilizes isolated human T cells that have been genetically-modified to enhance their specificity for a specific tumor associated antigen. Genetic modification may involve the expression of a chimeric antigen receptor (CAR) or an exogenous T cell receptor to graft antigen specificity onto the T cell. By contrast to exogenous T cell receptors, chimeric antigen receptors derive their specificity from the variable domains of a monoclonal antibody. Thus, T cells expressing chimeric antigen receptors (CAR T cells) induce tumor immunoreactivity in a major histocompatibility complex non-restricted manner. To date, T cell adoptive immunotherapy has been utilized as a clinical therapy for a number of cancers, including B cell malignancies (e.g., acute lymphoblastic leukemia (ALL), B cell non-Hodgkin lymphoma (NHL), and chronic lymphocytic leukemia), multiple myeloma, neuroblastoma, glioblastoma, advanced gliomas, ovarian cancer, mesothelioma, melanoma, and pancreatic cancer.

Currently, prior to CAR T cell therapy, patients are pre-treated with a round of chemotherapy for purposes of lymphodepletion. The lymphodepleting chemotherapeutic agents typically are fludarabine, cyclophosphamide, or a combination thereof. This is typically carried out 3 days to 1 week prior to injection with the CAR T cells. In terms of autologous cell therapy this is generally sufficient to eliminate enough of the host lymphocytes to make space for the incoming CAR T cells to benefit from the microenvironment of the host and promote expansion of the incoming CAR T cells (see Hay et al., Drugs 77(3) (2017)).

Treatment is more complicated with allogeneic CAR T cells because of the higher potential for host vs. graft rejection of the injected CAR T cells. Insufficient lymphodepletion can cause the host to elicit an immune response against the CAR T cells and limit their ability to expand and limit efficacy. One approach to overcoming this problem is to utilize a biologic or other agent that targets host immune cells but does not target the CAR T cell. Poirot et al., describes an approach where CART cells were engineered using TALENs to generate cells deficient in both the αβ T cell receptor and a second protein CD52, which is expressed on host lymphocytes (see Poirot et al., Cancer Research (18)75 (2015). The authors then utilized an anti-CD52 antibody to further deplete host lymphocytes. However, there are drawbacks to this approach because CD52 antibodies (e.g., alemtuzumab) can exhibit toxicities and are used in severe autoimmune diseases, such as relapsing multiple sclerosis and during immune ablative therapy prior to bone marrow transplantation (see Poire and Besien, Expert Opin Biol Ther. (8)11, 2012). In addition, CD52 antibodies are known to have a long half-life. Thus, patients are at higher risk of developing severe and prolonged cytopenias.

Accordingly, there is an unmet need for pre-treatment lymphodepletion regimens for preventing host vs. graft rejection of allogeneic cellular immunotherapies.

### SUMMARY OF THE INVENTION

Provided herein are methods and compositions for lymphodepletion in a subject in need thereof prior to or during treatment with a cellular immunotherapy comprising genetically-modified cells that lack CD3 on the cell surface and express one or more polypeptides of interest (e.g., a chimeric antigen receptor or an exogenous T cell receptor (TCR)). Further provided herein are compositions and methods for the treatment of a disease, such as cancer, with the genetically-modified cells disclosed herein in a subject who has undergone the lymphodepletion regimens of the invention. The present invention is based, in part, on the discovery that administration of an antibody that specifically binds an antigen (e.g., CD3) that is not present on the cell surface of a cellular immunotherapy according to the invention, but which is expressed on host lymphocytes, aids in the depletion of host immune cells while leaving the cellular immunotherapy unaffected. The present compositions and methods for lymphodepletion are useful to decrease the likelihood or severity of host vs. graft rejection of a cellular immunotherapeutic, while also allowing for expansion of the incoming cellular immunotherapeutic.

Thus, in one aspect, the invention provides a method of immunotherapy for treating a cancer in a subject in need thereof, the method comprising administering to the subject an antibody, or antigen-binding fragment thereof, that specifically binds CD3 in an amount effective to deplete a population of lymphocytes in the subject; and administering to the subject a composition comprising a population of genetically-modified T cells that have no detectable CD3 on the cell surface, wherein the population of genetically-modified T cells comprise in their genome an exogenous polynucleotide encoding a chimeric antigen receptor (CAR) or an exogenous T cell receptor (TCR) that is expressed by the genetically-modified T cells.

In one embodiment, the method further comprises administering a lymphodepleting chemotherapeutic agent or an additional lymphodepleting antibody to the subject prior to administration of the composition comprising the population of genetically-modified T cells.

In some such embodiments, the antibody, or antigen binding fragment thereof, is administered to the subject prior to administration of the composition comprising the population of genetically-modified T cells. In other such embodiments, the antibody, or antigen binding fragment thereof, is administered to the subject concomitant with administration of the composition comprising the population of genetically-modified T cells. In yet other such embodiments, the antibody, or antigen binding fragment thereof, is administered to the subject following administration of the composition comprising the population of genetically-modified T cells.

In another aspect, the invention provides a method of immunotherapy for treating a cancer in a subject in need thereof, the method comprising administering to the subject a composition comprising a population of genetically-modified T cells that have no detectable CD3 on the cell surface, wherein the population of genetically-modified T cells comprise in their genome an exogenous polynucleotide encoding a chimeric antigen receptor (CAR) or an exogenous T cell receptor (TCR) that is expressed by the genetically-modified cells; and wherein the subject has previously been administered a lymphodepleting chemotherapeutic agent and an antibody or antigen-binding fragment thereof that specifically binds CD3 in an amount effective to deplete a population of lymphocytes in the subject.

In some embodiments of a method provided herein, the antibody, or antigen binding fragment thereof, is administered to the subject 1-30 days prior to administration of the population of genetically-modified T cells.

In some embodiments, the antibody, or antigen binding fragment thereof, is administered to the subject prior to administration of the lymphodepleting chemotherapeutic agent. In other embodiments, the antibody, or antigen binding fragment thereof, is administered to the subject concomitant with administration of the lymphodepleting chemotherapeutic agent. In yet further embodiments, the antibody, or antigen binding fragment thereof, is administered to the subject following administration of the chemotherapeutic agent.

In some embodiments, the antibody, or antigen binding fragment thereof, is administered to the subject at a dose of from about 0.01 mg/kg to about 1.0 mg/kg.

In some embodiments, the antibody, or antigen-binding fragment thereof, is selected from the group consisting of a monoclonal antibody, a polyclonal antibody, a humanized antibody, a fully human antibody, a bispecific antibody, a dual-variable immunoglobulin domain, a single-chain Fv molecule (scFv), a sdAb, a diabody, a triabody, a nanobody, an antibody-like protein scaffold, a Fv fragment, a Fab fragment, a F(ab′)₂ molecule, and a tandem di-scFv.

In some embodiments, the antibody, or antigen-binding fragment thereof, does not detectably bind the genetically-modified T cells.

In some embodiments, the composition comprising the population of genetically-modified T cells is administered to the subject at a dose of 1×10³ to 1×10⁹ genetically-modified cells/kg.

In some embodiments, the lymphodepleting chemotherapeutic agent is administered three or more days prior to administration of the composition comprising the population of genetically-modified T cells.

In some embodiments, the lymphodepleting chemotherapeutic agent is administered seven days or less prior to administration of the composition comprising the population of genetically-modified T cells.

In some embodiments, the lymphodepleting chemotherapeutic agent is fludarabine, cyclophosphamide, bendamustine, melphalan, 6-mercaptopurine (6-MP), daunorubicin, cytarabine, L-asparaginase, methotrexate, prednisone, dexamethasone, nelarabine, and the additional lymphodepleting antibody is an anti-CD52 antibody (e.g., alemtuzumab) or rituximab or a combination thereof.

In some embodiments, cyclophosphamide is administered to the subject at a dose of about 250-1500 mg/m²/day. In certain embodiments, cyclophosphamide is administered to the subject at a dose of about 500-1000 mg/m²/day. In some embodiments, the dose of cyclophosphamide is about 250-1500 mg/m²/day, about 300-1500 mg/m²/day, about 350-1500 mg/m²/day, about 400-1500 mg/m²/day, about 450-1500 mg/m²/day, about 500-1500 mg/m²/day, about 550-1500 mg/m²/day, or about 600-1500 mg/m²/day. In another embodiment, the dose of cyclophosphamide is about 250-1500 mg/m²/day, about 350-1000 mg/m²/day, about 400-900 mg/m²/day, about 450-800 mg/m²/day, about 450-700 mg/m²/day, about 450-600 mg/m²/day, or about 450-550 mg/m²/day. In some embodiments, the dose of cyclophosphamide is about 250 mg/m²/day, about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day.

In certain embodiments, cyclophosphamide is administered to the subject at a dose of about 500 mg/m²/day. In another embodiment, cyclophosphamide is administered to the subject at a dose of about 500 mg/m²/day daily starting five days and ending two to three days prior to administration of the composition comprising the population of genetically-modified T cells. In another embodiment, cyclophosphamide is administered to the subject at a dose of about 500 mg/m²/day daily starting five days and ending two days prior to administration of the composition comprising the population of genetically-modified T cells. In one such embodiment, cyclophosphamide is administered to the subject at a dose of about 500 mg/m²/day daily starting five days and ending three days prior to administration of the composition comprising the population of genetically-modified T cells.

In certain embodiments, cyclophosphamide is administered to the subject at a dose of about 1000 mg/m²/day. In one such embodiment, cyclophosphamide is administered to the subject at a dose of about 1000 mg/m²/day daily starting four days and ending two to three days prior to administration of the composition comprising the population of genetically-modified T cells. In one such embodiment, cyclophosphamide is administered to the subject at a dose of about 1000 mg/m²/day daily starting four days and ending two days prior to administration of the composition comprising the population of genetically-modified T cells. In one such embodiment, cyclophosphamide is administered to the subject at a dose of about 1000 mg/m²/day daily starting four days and ending three days prior to administration of the composition comprising the population of genetically-modified T cells.

In some embodiments, fludarabine is administered to the subject at a dose of 10-40 mg/m²/day. In some embodiments, the dose of fludarabine is about 10-100 mg/m²/day, about 15-100 mg/m²/day, about 20-100 mg/m²/day, about 25-900 mg/m²/day, about 30-900 mg/m²/day, about 35-100 mg/m²/day, about 40-100 mg/m²/day, about 45-100 mg/m²/day, about 50-100 mg/m²/day, about 55-100 mg/m²/day, or about 60-100 mg/m²/day. In other embodiments, the dose of fludarabine is about 10-100 mg/m²/day, about 10-90 mg/m²/day, about 10-80 mg/m²/day, about 10-70 mg/m²/day, about 10-60 mg/m²/day, about 10-50 mg/m²/day, about 10-45 mg/m²/day, about 20-40 mg/m²/day, about 25-35 mg/m²/day, or about 28-32 mg/m²/day. In certain embodiments, the dose of fludarabine is about 10 mg/m²/day, 15 mg/m²/day, 20 mg/m²/day, 25 mg/m²/day, 30 mg/m²/day, 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day.

In certain embodiments, fludarabine is administered to the subject at a dose of 30 mg/m²/day. In another embodiment, fludarabine is administered to the subject at a dose of about 30 mg/m²/day daily starting five days and ending two to three days prior to administration of the composition comprising the population of genetically-modified T cells. In another embodiment, fludarabine is administered to the subject at a dose of about 30 mg/m²/day daily starting five days and ending two days prior to administration of the composition comprising the population of genetically-modified T cells. In one such embodiment, fludarabine is administered to the subject at a dose of about 30 mg/m²/day daily starting five days and ending three days prior to administration of the composition comprising the population of genetically-modified T cells. In another such embodiment, fludarabine is administered to the subject at a dose of about 30 mg/m²/day daily starting seven days and ending two to three days prior to administration of the composition comprising the population of genetically-modified T cells. In another such embodiment, fludarabine is administered to the subject at a dose of about 30 mg/m²/day daily starting seven days and ending two days prior to administration of the composition comprising the population of genetically-modified T cells. In another such embodiment, fludarabine is administered to the subject at a dose of about 30 mg/m²/day daily starting seven days and ending three days prior to administration of the composition comprising the population of genetically-modified T cells.

In some embodiments, the lymphodepleting chemotherapeutic agent is administered in combination with an additional cancer therapy selected from the group consisting of an additional lymphodepleting chemotherapeutic agent, surgery, radiation, and gene therapy.

In some embodiments, the CAR or the exogenous TCR specifically binds to a molecule on the surface of a cancer cell. In certain embodiments, the chimeric antigen receptor specifically binds to CD19, CD20, BCMA, CLL1, CS1 (SLAMF7), MUC1, FLT3, HPV16 E6, or HPV16 E7.

In some embodiments, the exogenous polynucleotide is within a target gene in the genome of the genetically-modified T cell. In certain embodiments, the target gene is selected from the group consisting of a TCR alpha gene, a TCR alpha constant (TRAC) gene, a TCR beta gene, or a TCR beta constant (TRBC) gene.

In some embodiments, the genetically-modified T cells have no detectable cell surface expression of an endogenous T cell receptor.

In some embodiments, the genetically-modified T cell is a human T cell, or a cell derived therefrom.

In some embodiments, the cancer is selected from the group consisting of a cancer of carcinoma, lymphoma, sarcoma, blastomas, myeloma, and leukemia.

In some embodiments, the cancer is selected from the group consisting of lung cancer, melanoma, breast cancer, prostate cancer, colon cancer, renal cell carcinoma, ovarian cancer, neuroblastoma, rhabdomyosarcoma, multiple myeloma, leukemia, lymphoma, acute lymphoblastic leukemia, small cell lung cancer, Hodgkin's lymphoma, and childhood acute lymphoblastic leukemia.

In some embodiments, the cancer is selected from the group consisting of a cancer of B-cell origin. In certain embodiments, the cancer of B-cell origin is selected from the group consisting of B-lineage acute lymphoblastic leukemia, B-cell chronic lymphocytic leukemia, multiple myeloma, and B-cell non-Hodgkin's lymphoma.

In some embodiments, the subject is a human subject.

In some embodiments, the method is effective to treat or reduce the symptoms of the cancer.

In some embodiments, the method is effective to treat or prevent host-vs-graft disease.

In some embodiments, the immunotherapy is an allogeneic cellular immunotherapy.

In some embodiments, the genetically-modified T cells are generated by inserting an exogenous polynucleotide encoding the CAR or the exogenous TCR within a chromosome of a T cell by a method comprising transfecting the T cell with one or more nucleic acids including: (a) a first nucleic acid comprising a polynucleotide encoding an engineered nuclease having specificity for a recognition sequence within the chromosome, wherein the engineered nuclease is expressed in the T cell; and (b) a template nucleic acid comprising the exogenous polynucleotide; wherein the engineered nuclease generates a cleavage site within the chromosome at the recognition sequence, and wherein the exogenous polynucleotide encoding the CAR or the exogenous TCR is inserted into the chromosome at the cleavage site.

In some embodiments, the template nucleic acid is introduced into the T cell using a viral vector. In certain embodiments, the viral vector is a recombinant AAV vector.

In some embodiments, the engineered nuclease is an engineered meganuclease, a zinc finger nuclease, a TALEN, a compact TALEN, a CRISPR system nuclease, or a megaTAL.

In some embodiments, the engineered nuclease is an engineered meganuclease.

In another aspect, provided herein is a kit comprising: (a) an antibody, or antigen-binding fragment thereof, that specifically binds CD3; and (b) a composition comprising a population of genetically-modified T cells, wherein the population of genetically-modified T cells comprise in their genome an exogenous polynucleotide encoding a chimeric antigen receptor (CAR) or an exogenous T cell receptor (TCR) that is expressed by the genetically-modified T cells.

In another aspect, provided herein is a kit comprising: (a) a lymphodepleting chemotherapeutic agent, (b) an antibody, or antigen-binding fragment thereof, that specifically binds CD3; and (c) a composition comprising a population of genetically-modified T cells, wherein the population of genetically-modified T cells comprise in their genome an exogenous polynucleotide encoding a chimeric antigen receptor (CAR) or an exogenous T cell receptor (TCR) that is expressed by the genetically-modified T cells.

In some embodiments of a kit provided herein, the lymphodepleting chemotherapeutic agent is fludarabine, cyclophosphamide, or a combination thereof.

In some embodiments, the exogenous polynucleotide is within a target gene in the genome of the genetically-modified T cell. In certain embodiments, the target gene is selected from the group consisting of TCR alpha gene, a TCR alpha constant (TRAC) gene, a TCR beta gene, or a TCR beta constant (TCBC) gene.

In some embodiments, the genetically-modified T cell is a human T cell, or a cell derived therefrom.

In some embodiments, the CAR or the exogenous TCR specifically binds to a molecule on the surface of a cancer cell. In certain embodiments, the CAR specifically binds to CD19, CD20, BCMA, or CLL1.

In some embodiments, the kit further comprises instructions for use of components of the kit in treating a cancer.

In another aspect, the invention provides a method for reducing the number of target cells in a subject, wherein the method comprises: (a) administering to the subject a lymphodepletion regimen that comprises administering one or more effective doses of an antibody, or antigen-binding fragment thereof, that specifically binds CD3; and (b) administering to the subject an effective dose of a pharmaceutical composition comprising a population of human immune cells, wherein a plurality of the human immune cells are genetically-modified human immune cells that express a CAR or an exogenous TCR; wherein the CAR or the exogenous TCR comprises an extracellular ligand-binding domain having specificity for an antigen on the target cells.

In some embodiments, the genetically-modified human immune cells comprise an inactivated TCR alpha gene. In some embodiments, the genetically-modified human immune cells comprise an inactivated TCR alpha constant region (TRAC) gene. In some embodiments, the genetically-modified human immune cells comprise an inactivated TCR beta gene. In some embodiments, the genetically-modified human immune cells comprise an inactivated TCR beta constant region (TRBC) gene.

In some embodiments, the one or more effective doses of the antibody, or antigen-binding fragment thereof, depletes a population of endogenous lymphocytes in the subject.

In some embodiments, the antibody, or antigen-binding fragment thereof, is selected from the group consisting of a monoclonal antibody, a polyclonal antibody, a humanized antibody, a fully human antibody, a bispecific antibody, a dual-variable immunoglobulin domain, a single-chain Fv molecule (scFv), a sdAb, a diabody, a triabody, a nanobody, an antibody-like protein scaffold, a Fv fragment, a Fab fragment, a F(ab′)2 molecule, and a tandem di-scFv.

In some embodiments, the antibody, or antigen-binding fragment thereof, does not detectably bind the genetically-modified human immune cells.

In some embodiments, the antibody, or antigen binding fragment thereof, is administered to the subject prior to administration of the pharmaceutical composition. In certain embodiments, the antibody, or antigen binding fragment thereof, is administered to the subject concurrently with administration of the pharmaceutical composition. In certain embodiments, the antibody, or antigen binding fragment thereof, is administered to the subject following administration of the pharmaceutical composition.

In some embodiments, the dosage of anti-CD3 antibody is about 0.001 mg/kg. In some embodiments, the dosage of anti-CD3 antibody is about 0.005 mg/kg. In some embodiments, the dosage of anti-CD3 antibody is about 0.01 mg/kg. In some embodiments, the dosage of anti-CD3 antibody is about 0.05 mg/kg. In some embodiments, the dosage of anti-CD3 antibody is about 0.1 mg/kg. In some embodiments, the dosage of anti-CD3 antibody is about 0.5 mg/kg. In some embodiments, the dosage of anti-CD3 antibody is about 1 mg/kg. In some embodiments, the dosage of anti-CD3 antibody is about 2.5 mg/kg. In some embodiments, the dosage of anti-CD3 antibody is about 5 mg/kg. In some embodiments, the dosage of anti-CD3 antibody is about 10 mg/kg. In some embodiments, the dosage of anti-CD3 antibody is about 15 mg/kg. In some embodiments, the dosage of anti-CD3 antibody is about 20 mg/kg. In some embodiments, the dosage of anti-CD3 antibody is about 25 mg/kg. In some embodiments, the dosage of anti-CD3 antibody is about 30 mg/kg. In some embodiments, the dosage of anti-CD3 antibody is about 35 mg/kg. In some embodiments, the dosage of anti-CD3 antibody is about 40 mg/kg. In some embodiments, the dosage of anti-CD3 antibody is about 45 mg/kg. In some embodiments, the dosage of anti-CD3 antibody is about 50 mg/kg. In some embodiments, the dosage of anti-CD3 antibody is about 60 mg/kg. In some embodiments, the dosage of anti-CD3 antibody is about 70 mg/kg. In some embodiments, the dosage of anti-CD3 antibody is about 80 mg/kg. In some embodiments, the dosage of anti-CD3 antibody is about 90 mg/kg. In some embodiments, the dosage of anti-CD3 antibody is about 100 mg/kg. In some embodiments, the dosage of anti-CD3 antibody is about 110 mg/kg. In some embodiments, the dosage of anti-CD3 antibody is about 120 mg/kg. In some embodiments, the dosage of anti-CD3 antibody is about 130 mg/kg. In some embodiments, the dosage of anti-CD3 antibody is about 140 mg/kg. In some embodiments, the dosage of anti-CD3 antibody is about 150 mg/kg.

In some embodiments, the antibody, or antigen binding fragment thereof, is administered to the subject 1-30 days prior to administration of the pharmaceutical composition.

In some embodiments, the antibody, or antigen binding fragment thereof, is administered to the subject within 10 days prior to administration of the pharmaceutical composition.

In some embodiments, the anti-CD3 antibody, or antigen binding fragment thereof, is administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more prior to administration of the pharmaceutical composition.

In some embodiments, the anti-CD3 antibody, or antigen binding fragment thereof, is administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more following administration of the pharmaceutical composition.

In some embodiments, the antibody, or antigen binding fragment thereof, is administered intravenously. In some embodiments, the antibody, or antigen binding fragment thereof, is administered orally. In some embodiments, the antibody, or antigen binding fragment thereof, is administered subcutaneously.

In some embodiments, the pharmaceutical composition is administered at a dose of between about 1×10⁴ and about 1×10⁸ genetically-modified human immune cells/kg. In some embodiments, the pharmaceutical composition is administered at a dose of between about 1×10⁵ and about 1×10⁷ genetically-modified human immune cells/kg. In some embodiments, the pharmaceutical composition is administered at a dose of between about 1×10⁵ and about 6×10⁶ genetically-modified human immune cells/kg. In some embodiments, the pharmaceutical composition is administered at a dose of between about 3×10⁵ and about 6×10⁶ genetically-modified human immune cells/kg. In some embodiments, the pharmaceutical composition is administered at a dose of between about 3×10⁵ and about 3×10⁶ genetically-modified human immune cells/kg. In some embodiments, the pharmaceutical composition is administered at a dose of about 0.5×10⁶ genetically-modified human immune cells/kg. In some embodiments, the pharmaceutical composition is administered at a dose of about 1.0×10⁶ genetically-modified human immune cells/kg. In some embodiments, the pharmaceutical composition is administered at a dose of about 2.0×10⁶ genetically-modified human immune cells/kg. In some embodiments, the pharmaceutical composition is administered at a dose of about 3.0×10⁶ genetically-modified human immune cells/kg. In some embodiments, the dose of the pharmaceutical composition comprises no more than 3×10⁸ genetically-modified human immune cells.

In some embodiments, the method further comprises administering a second dose of the pharmaceutical composition to the subject.

In some embodiments, the method further comprises administering one or more effective doses of one or more lymphodepleting agents to the subject prior to administration of the pharmaceutical composition.

In some embodiments, the lymphodepleting agent is administered to the subject prior to administration of the antibody, or antigen-binding fragment thereof, and prior to administration of the pharmaceutical composition. In certain embodiments, the lymphodepleting agent is administered to the subject concurrently with administration of the antibody, or antigen-binding fragment thereof, and prior to administration of the pharmaceutical composition. In some embodiments, the lymphodepleting agent is administered to the subject following administration of the antibody, or antigen-binding fragment thereof, and prior to administration of the pharmaceutical composition.

In some embodiments, the lymphodepleting agent is fludarabine, cyclophosphamide, bendamustine, melphalan, 6-mercaptopurine (6-MP), daunorubicin, cytarabine, L-asparaginase, methotrexate, prednisone, dexamethasone, nelarabine, or a combination thereof.

In certain embodiments, the lymphodepleting agent is cyclophosphamide. In some embodiments, cyclophosphamide is administered to the subject at a dose of about 250-1500 mg/m²/day. In certain embodiments, cyclophosphamide is administered to the subject at a dose of about 500-1000 mg/m²/day. In some embodiments, the dose of cyclophosphamide is about 250-1500 mg/m²/day, about 300-1500 mg/m²/day, about 350-1500 mg/m²/day, about 400-1500 mg/m²/day, about 450-1500 mg/m²/day, about 500-1500 mg/m²/day, about 550-1500 mg/m²/day, or about 600-1500 mg/m²/day. In another embodiment, the dose of cyclophosphamide is about 250-1500 mg/m²/day, about 350-1000 mg/m²/day, about 400-900 mg/m²/day, about 450-800 mg/m²/day, about 450-700 mg/m²/day, about 450-600 mg/m²/day, or about 450-550 mg/m²/day. In some embodiments, the dose of cyclophosphamide is about 250 mg/m²/day, about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day.

In some embodiments, cyclophosphamide is administered to the subject at a dose of about 500-1000 mg/m²/day. In some embodiments, cyclophosphamide is administered to the subject at a dose of about 500 mg/m²/day. In some embodiments, cyclophosphamide is administered to the subject at a dose of about 500 mg/m²/day daily starting five days and ending two days prior to administration of the pharmaceutical composition. In some embodiments, cyclophosphamide is administered to the subject at a dose of about 500 mg/m²/day daily starting five days and ending three days prior to administration of the pharmaceutical composition. In some embodiments, cyclophosphamide is administered to the subject at a dose of about 1000 mg/m²/day. In some embodiments, cyclophosphamide is administered to the subject at a dose of about 1000 mg/m²/day daily starting four days and ending two days prior to administration of the pharmaceutical composition. In some embodiments, cyclophosphamide is administered to the subject at a dose of about 1000 mg/m²/day daily starting four days and ending three days prior to administration of the pharmaceutical composition.

In certain embodiments, the lymphodepleting agent is fludarabine. In some embodiments, fludarabine is administered to the subject at a dose of 10-40 mg/m²/day. In some embodiments, the dose of fludarabine is about 10-100 mg/m²/day, about 15-100 mg/m²/day, about 20-100 mg/m²/day, about 25-900 mg/m²/day, about 30-900 mg/m²/day, about 35-100 mg/m²/day, about 40-100 mg/m²/day, about 45-100 mg/m²/day, about 50-100 mg/m²/day, about 55-100 mg/m²/day, or about 60-100 mg/m²/day. In other embodiments, the dose of fludarabine is about 10-100 mg/m²/day, about 10-90 mg/m²/day, about 10-80 mg/m²/day, about 10-70 mg/m²/day, about 10-60 mg/m²/day, about 10-50 mg/m²/day, about 10-45 mg/m²/day, about 20-40 mg/m²/day, about 25-35 mg/m²/day, or about 28-32 mg/m²/day. In certain embodiments, the dose of fludarabine is about 10 mg/m²/day, 15 mg/m²/day, 20 mg/m²/day, 25 mg/m²/day, 30 mg/m²/day, 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day. In some embodiments, fludarabine is administered to the subject at a dose of 30 mg/m²/day. In some embodiments, fludarabine is administered to the subject at a dose of about 30 mg/m²/day daily starting five days and ending two days prior to administration of the pharmaceutical composition. In some embodiments, fludarabine is administered to the subject at a dose of about 30 mg/m²/day daily starting five days and ending three days prior to administration of the pharmaceutical composition. In some embodiments, fludarabine is administered to the subject at a dose of about 30 mg/m²/day daily starting seven days and ending two to three days prior to administration of the pharmaceutical composition.

In some embodiments, the lymphodepletion regimen does not comprise administering the subject an effective dose of a biological lymphodepletion agent. In certain embodiments, the lymphodepletion regimen does not comprise administering the subject a biological lymphodepletion agent. In some embodiments, the lymphodepletion regimen comprises administering the subject one or more effective doses of a biological lymphodepletion agent. In certain embodiments, the biological lymphodepletion agent is a monoclonal antibody, or a fragment thereof. In some embodiments, the monoclonal antibody, or fragment thereof, has specificity for a T cell antigen. In some embodiments, the monoclonal antibody, or fragment thereof, is an anti-CD52 monoclonal antibody. In some embodiments, the monoclonal antibody is alemtuzumab or ALLO-647.

In some embodiments, the biological lymphodepletion agent is administered to the subject prior to administration of the antibody, or antigen-binding fragment thereof, and prior to administration of the pharmaceutical composition. In certain embodiments, the biological lymphodepletion agent is administered to the subject concurrently with administration of the antibody, or antigen-binding fragment thereof, and prior to administration of the pharmaceutical composition. In some embodiments, the biological lymphodepletion agent is administered to the subject following administration of the antibody, or antigen-binding fragment thereof, and prior to administration of the pharmaceutical composition.

In some embodiments, the subject is administered an additional therapy selected from the group consisting of an additional chemotherapeutic agent, surgery, radiation, and gene therapy.

In some embodiments, a transgene encoding the CAR or the exogenous TCR is inserted into the genome of the genetically-modified human immune cells within the TCR alpha gene, the TRAC gene, the TCR beta gene, or the TRBC gene, wherein the transgene disrupts expression of the TCR alpha gene, the TRAC gene, the TCR beta gene, or the TRBC gene. In some embodiments, the transgene encoding the CAR or the exogenous TCR is inserted into the TRAC gene. In certain embodiments, the transgene encoding the CAR or the exogenous TCR is inserted into a recognition sequence for an engineered meganuclease, a TALEN, a compact TALEN, a zinc finger nuclease, a megaTAL, or a CRISPR system nuclease. In certain embodiments, the transgene encoding the CAR or the exogenous TCR is inserted into an engineered meganuclease recognition sequence comprising SEQ ID NO: 1 within the TRAC gene. In particular embodiments, the transgene encoding the CAR or the exogenous TCR is inserted between positions 13 and 14 of SEQ ID NO: 1 within the TRAC gene.

In some embodiments, the genetically-modified human immune cells do not have detectable cell surface expression of an endogenous alpha/beta TCR.

In some embodiments, the genetically-modified human immune cells do not have detectable cell surface expression of CD3.

In some embodiments, the human immune cells are derived from the subject. In certain embodiments, the human immune cells are not derived from the subject.

In some embodiments, the method is effective to treat or reduce the symptoms of the cancer.

In some embodiments, the method is effective to treat or prevent host-vs-graft disease.

In some embodiments, the immunotherapy is an allogeneic cellular immunotherapy.

In some embodiments, the human immune cells are human T cells, or cells derived therefrom, or human natural killer (NK) cells, or cells derived therefrom. In certain embodiments, the human immune cells are human T cells.

In some embodiments, the target cells are cancer cells. In some embodiments, the cancer cells are blood cancer cells. In some embodiments, the cancer cells are from a solid tumor.

In some embodiments, the number of target cells is reduced. In some embodiments, the method reduces the size of the cancer in the subject. In some embodiments, the method eradicates the cancer in the subject.

In some embodiments, the cancer cells are from a cancer of B cell origin or multiple myeloma. In certain embodiments, the cancer of B cell origin is acute lymphoblastic leukemia (ALL), chronic lymphocytic leukemia (CLL), small lymphocytic lymphoma (SLL), or non-Hodgkin lymphoma (NHL). In some embodiments, the NHL is mantle cell lymphoma (MCL) or diffuse large B cell lymphoma (DLBCL). In some embodiments, the cancer cells are from a solid tumor cancer.

In some embodiments, the subject is refractory to prior immunotherapy. In certain embodiments, the subject is refractory to prior CAR T cell immunotherapy. In certain embodiments the subject is refractory to prior CAR NK cell immunotherapy. In certain embodiments, the subject is refractory to prior exogenous TCR/T cell immunotherapy. In certain embodiments the subject is refractory to prior exogenous TCR/NK cell immunotherapy.

In some embodiments, the genetically-modified human immune cell comprises a CAR, wherein the extracellular ligand-binding domain of the CAR comprises a single-chain variable fragment (scFv). In some embodiments, the CAR comprises a CD8 alpha hinge domain (SEQ ID NO: 11). In some embodiments, the CAR comprises a CD8 alpha transmembrane domain (SEQ ID NO: 10). In some embodiments, the CAR comprises a co-stimulatory domain comprising one or more TRAF-binding domains. In certain embodiments, the CAR comprises a co-stimulatory domain comprising a first domain comprising SEQ ID NO: 3 and a second domain comprising SEQ ID NO: 4 or 5. In certain embodiments, the CAR comprises a novel 6 (N6) co-stimulatory domain (SEQ ID NO: 6) or a 4-1BB co-stimulatory domain (SEQ ID NO: 7). In certain embodiments, the CAR comprises CD3 zeta intracellular signaling domain (SEQ ID NO: 8).

In some embodiments, the genetically-modified human immune cells represent between about 40% and about 75% of the human immune cells in the population. In particular embodiments, the genetically-modified human immune cells represent between about 50% and about 70% of the human immune cells in the population.

In some embodiments, the genetically-modified human immune cells proliferate in vivo for at least one day following administration of the pharmaceutical composition. In certain embodiments, the genetically-modified human immune cells proliferate in vivo between about day 1 and about day 21 following administration of the pharmaceutical composition. In certain embodiments, the number of copies of the CAR or the exogenous TCR transgene per mg of DNA in peripheral blood mononuclear cells is elevated for up to 21 days after administration of the pharmaceutical composition when compared to the number of copies present prior to administration.

In some embodiments, the serum concentration of C-reactive protein, ferritin, IL-6, interferon gamma, or any combination thereof, is elevated compared to the concentration at day 0 for at least 1 day following administration of the pharmaceutical composition.

In some embodiments, the method is an immunotherapy for the treatment of a disease, such as cancer, wherein the subject achieves a partial response or a complete response to the method of immunotherapy. In certain embodiments, the partial response or the complete response is maintained through at least 28 days after administration of the pharmaceutical composition.

In another aspect, the invention provides a method of killing a population of target cells, wherein the method comprises contacting the population of target cells with a population of human immune cells, wherein the population of human immune cells comprises a plurality of genetically-modified human immune cells expressing a CAR or an exogenous TCR, wherein the CAR or the exogenous TCR has specificity for an antigen present on the target cells, and wherein the genetically-modified human immune cells comprise an inactivated gene encoding a component of the endogenous alpha/beta TCR, and wherein the target cells are contacted with the population of human immune cells in the presence of an antibody, or antigen binding fragment thereof, that specifically binds CD3.

In some embodiments, the antibody, or antigen binding fragment thereof, is a monoclonal antibody, or antigen binding fragment thereof. In some embodiments, the antibody, or antigen binding fragment thereof, is a monoclonal antibody, or antigen binding fragment thereof.

In some embodiments, the inactivated gene is a TCR alpha gene. In some embodiments, the inactivated gene is a TRAC gene. In some embodiments, the inactivated gene is a TCR beta gene. In some embodiments, the inactivated gene is a TRBC gene.

In some embodiments, the genetically-modified human immune cells have no detectable cell surface expression of endogenous CD3. In some embodiments, the genetically-modified human immune cells have no detectable cell surface expression of an endogenous alpha/beta TCR.

In some embodiments, the antibody, or antigen binding fragment thereof, does not bind to the genetically-modified human immune cells. In some embodiments, the antibody, or antigen binding fragment thereof, does not kill said genetically-modified human immune cells.

In some embodiments, a transgene encoding the CAR or the exogenous TCR is inserted into the genome of the genetically-modified human immune cells within the TCR alpha gene, the TRAC gene, the TCR beta gene, or the TRBC gene, wherein the transgene disrupts expression of the TCR alpha gene, the TRAC gene, the TCR beta gene, or the TRBC gene. In some embodiments, the transgene encoding the CAR or the exogenous TCR is inserted into the TRAC gene. In certain embodiments, the transgene encoding the CAR or the exogenous TCR is inserted into a recognition sequence for an engineered meganuclease, a TALEN, a compact TALEN, a zinc finger nuclease, a megaTAL, or a CRISPR system nuclease. In certain embodiments, the transgene encoding the CAR or the exogenous TCR is inserted into an engineered meganuclease recognition sequence comprising SEQ ID NO: 1 within the TRAC gene. In particular embodiments, the transgene encoding the CAR or the exogenous TCR is inserted between positions 13 and 14 of SEQ ID NO: 1 within the TRAC gene.

In some embodiments, the human immune cells are derived from the same subject as the target cells. In certain embodiments, the human immune cells are not derived from the same subject as the target cells.

In some embodiments, the human immune cells are human T cells, or cells derived therefrom, or human natural killer (NK) cells, or cells derived therefrom. In certain embodiments, the human immune cells are human T cells.

In some embodiments, the target cells are cancer cells.

In some embodiments, the genetically-modified human immune cell comprises a CAR, wherein the extracellular ligand-binding domain of the CAR comprises a single-chain variable fragment (scFv). In some embodiments, the CAR comprises a CD8 alpha hinge domain (SEQ ID NO: 11). In some embodiments, the CAR comprises a CD8 alpha transmembrane domain (SEQ ID NO: 10). In some embodiments, the CAR comprises a co-stimulatory domain comprising one or more TRAF-binding domains. In certain embodiments, the CAR comprises a co-stimulatory domain comprising a first domain comprising SEQ ID NO: 3 and a second domain comprising SEQ ID NO: 4 or 5. In certain embodiments, the CAR comprises a novel 6 (N6) co-stimulatory domain (SEQ ID NO: 6) or a 4-1BB co-stimulatory domain (SEQ ID NO: 7). In certain embodiments, the CAR comprises CD3 zeta intracellular signaling domain (SEQ ID NO: 8).

In some embodiments, the genetically-modified human immune cells represent between about 40% and about 75% of the human immune cells in the population. In particular embodiments, the genetically-modified human immune cells represent between about 50% and about 70% of the human immune cells in the population.

In some embodiments, the ratio of genetically-modified human immune cells to target cells is 10:1. In some embodiments, the ratio of genetically-modified human immune cells to target cells is 8:1. In some embodiments, the ratio of genetically-modified human immune cells to target cells is 6:1. In some embodiments, the ratio of genetically-modified human immune cells to target cells is 4:1. In some embodiments, the ratio of genetically-modified human immune cells to target cells is 2:1. In some embodiments, the ratio of genetically-modified human immune cells to target cells is 1:1. In some embodiments, the ratio of genetically-modified human immune cells to target cells is 1:2. In some embodiments, the ratio of genetically-modified human immune cells to target cells is 1:4. In some embodiments, the ratio of genetically-modified human immune cells to target cells is 1:6. In some embodiments, the ratio of genetically-modified human immune cells to target cells is 1:8. In some embodiments, the ratio of genetically-modified human immune cells to target cells is 1:10.

In some embodiments, the population of human immune cells and the target cells are contacted in vitro (i.e., ex vivo). In some embodiments, the population of human immune cells and the target cells are contacted in vivo within a subject. In some embodiments, the subject is administered a pharmaceutical composition comprising the population of human immune cells and a lymphodepletion regimen that includes one or more effective doses of the antibody, or antigen binding fragment thereof. In some embodiments, the target cells in the subject are cancer cells. In some embodiments, administration of the pharmaceutical composition and the lymphodepletion regimen are an immunotherapy for reducing the number of cancer cells in the subject.

In another aspect, the invention provides genetically-modified cells, or populations thereof, described herein for use as a medicament. The present disclosure further provides the use of genetically-modified cells, or populations thereof, described herein, in the manufacture of a medicament for treating a disease in a subject in need thereof. In some embodiments, the medicament is useful for treating cancer, such as a method of cancer immunotherapy, in a subject in need thereof.

In another aspect, the invention provides anti-CD3 antibodies, or antigen binding fragments thereof, described herein for use as a medicament. The present disclosure further provides the use of anti-CD3 antibodies, or antigen binding fragments thereof, described herein, in the manufacture of a medicament for treating a disease in a subject in need thereof. In some embodiments, the medicament is useful for treating cancer, such as a method of cancer immunotherapy, in a subject in need thereof.

In another aspect, the invention provides combinations of genetically-modified cells, or populations thereof, described herein, and anti-CD3 antibodies, or antigen binding fragments thereof, described herein for use as a medicament. The present disclosure further provides the use of genetically-modified cells, or populations thereof, described herein, and anti-CD3 antibodies, or antigen binding fragments thereof, described herein in the manufacture of a medicament for treating a disease in a subject in need thereof. In some embodiments, the medicament is useful for treating cancer, such as a method of cancer immunotherapy, in a subject in need thereof.

### BRIEF DESCRIPTION OF THE SEQUENCES

SEQ ID NO: 1 sets forth the nucleic acid sequence of the TRC 1-2 recognition sequence (sense strand).

SEQ ID NO: 2 sets forth the nucleic acid sequence of the TRC 1-2 recognition sequence (antisense strand).

SEQ ID NO: 3 sets forth the amino acid sequence of a TRAF-binding domain.

SEQ ID NO: 4 sets forth the amino acid sequence of a TRAF-binding domain.

SEQ ID NO: 5 sets forth the amino acid sequence of a TRAF-binding domain.

SEQ ID NO: 6 sets forth the amino acid sequence of a Novel 6 (N6) co-stimulatory domain.

SEQ ID NO: 7 sets forth the amino acid sequence of a 4-1BB co-stimulatory domain.

SEQ ID NO: 8 sets forth the amino acid sequence of a CD3 zeta intracellular signaling domain.

SEQ ID NO: 9 sets forth the amino acid sequence of a wild-type I-CreI homing endonuclease.

SEQ ID NO: 10 sets forth the amino acid sequence of a CD8 alpha transmembrane domain.

SEQ ID NO: 11 sets forth the amino acid sequence of a CD8 alpha hinge domain.

SEQ ID NO: 12 sets forth the amino acid sequence of the TRC 1-2L.1592 meganuclease.

### BRIEF DESCRIPTION OF THE DRAWINGS

FIG. 1. Provides a line graph showing the percent killing of either CD3⁻ CAR T cells or CD3⁺ T cells in a complement dependent cytotoxicity assay. FIG. 1A shows the percent killing of CAR T cells in the presence of increasing amounts of an equine anti-human thymocyte globulin antibody (ATGAM). FIG. 1B shows the percent killing of CAR T cells or CD3⁺ T cells in the presence of a CD3-specific antibody.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.” As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention.

As used herein, the term “endogenous” in reference to a nucleotide sequence or protein is intended to mean a sequence or protein that is naturally comprised within or expressed by a cell.

As used herein, the terms “nuclease” and “endonuclease” are used interchangeably to refer to naturally-occurring or engineered enzymes, which cleave a phosphodiester bond within a polynucleotide chain.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI (SEQ ID NO: 9), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two nuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, S1 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, Endl, I-BasI, I-TevII, I-TevIII, I-TwoI, MspI, MyaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the term “megaTAL” refers to a single-chain endonuclease comprising a transcription activator-like effector (TALE) DNA binding domain with an engineered, sequence-specific homing endonuclease.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the terms “template nucleic acid,” “donor template,” or “repair template” refer to a nucleic acid sequence that is desired to be inserted into a cleavage site within a cell's genome. Such template nucleic acids or donor templates can comprise, for example, a transgene, such as an exogenous transgene, which encodes a protein of interest (e.g., a CAR). The template nucleic acid or donor template can comprise 5′ and 3′ homology arms having homology to 5′ and 3′ sequences, respectively, that flank a cleavage site in the genome where insertion of the template is desired. Insertion can be accomplished, for example, by homology-directed repair (HDR).

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “target site” or “target sequence” refers to a region of the chromosomal DNA of a cell comprising a recognition sequence for a nuclease.

As used herein, the term “target gene” refers to a gene in the genome of a cell (e.g., T cell) in which an exogenous polynucleotide (e.g., encoding a chimeric antigen receptor) has been or can be inserted. In some embodiments, the target gene includes a target site or target sequence recognized by a nuclease. In some embodiments, the target gene is a gene encoding a component of an alpha/beta T cell receptor. In some embodiments, the target gene is within a T cell receptor alpha constant region (TRAC) gene.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells. As used herein, “disrupting a target sequence” refers to the introduction of a mutation (e.g., frameshift mutation) that interferes with the gene function and prevents expression and/or function of the polypeptide/expression product encoded thereby.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template into a gene can result in no expression of an encoded protein, expression of a truncated protein, and/or expression of a protein that does not retain its wild-type function.

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety, a transmembrane domain, and an intracellular domain, wherein the intracellular domain comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular domain of a chimeric antigen receptor can also comprise a naturally-occurring ligand for an antigen of interest, or a fragment of a naturally-occurring ligand which retains the ability to bind the antigen of interest.

The intracellular stimulatory domain can include one or more cytoplasmic signaling domains that transmit an activation signal to the immune effector cell following antigen binding. Such cytoplasmic signaling domains can include, without limitation, CD3ζ.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such TRAF binding-domains may include, for example, those set forth in SEQ ID NOs: 3-5. Such intracellular co-stimulatory domains can be any of those known in the art and can include, without limitation, those co-stimulatory domains disclosed in WO 2018/067697 including, for example, Novel 6 (“N6”; SEQ ID NO: 6). Further examples of co-stimulatory domains can include 4-1BB (CD137; SEQ ID NO: 7), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain (SEQ ID NO: 10). Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain (SEQ ID NO: 11).

As used herein, the terms “exogenous T cell receptor” or “exogenous TCR” refer to a TCR whose sequence is introduced into the genome of an immune effector cell (e.g., a human T cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest.

As used herein, the term “reduced expression” in reference to a target protein (i.e., an endogenously expressed protein) refers to any reduction in the expression of the endogenous protein by a genetically-modified cell when compared to a control cell. The term reduced can also refer to a reduction in the percentage of cells in a population of cells that express an endogenous protein targeted by an inhibitory nucleic acid compared to a population of control cells. Exemplary and non-limiting inhibitory nucleic acids may include, without limitation, a short hairpin RNA (shRNA), a small interfering RNA (siRNA), a hairpin siRNA, a microRNA (miRNA), or a precursor miRNA. Inhibitory nucleic acids can further include microRNA-adapted shRNAs. Such a reduction may be up to 5%, 10%, 20%, 30%, 40%, 50%, 60%, 70%, 80%, 90%, 95%, 96%, 97%, 98%, or up to 99%. It is understood that the term “reduced” encompasses a partial or incomplete knockdown of a target or endogenous protein, and is distinguished from a complete knockdown, such as that achieved by gene inactivation by a nuclease described herein.

As used herein with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, the terms “T cell receptor alpha gene” or “TCR alpha gene” are interchangeable and refer to the locus in a T cell which encodes the T cell receptor alpha subunit. The T cell receptor alpha can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” refers to the coding sequence of the T cell receptor alpha gene. The TCR alpha constant region includes the wild-type sequence, and functional variants thereof, identified by NCBI Gene ID NO. 28755.

As used herein, the term “T cell receptor beta gene” or “TCR beta gene” refers to the locus in a T cell which encodes the T cell receptor beta subunit. The T cell receptor beta gene can refer to NCBI Gene ID number 6957.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a viral vector. Viral vectors can include, without limitation, retroviruses (i.e., retroviral vectors), lentiviruses (i.e., lentiviral vectors), adenoviruses (i.e., adenoviral vectors), and adeno-associated viruses (i.e., AAV vectors).

The term “antibody” as used herein in encompasses various antibody structures, including but not limited to antibodies from animal species (e.g., camelid antibodies, goat antibodies, murine antibodies, rabbit antibodies, and the like), humanized antibodies, monoclonal antibodies, polyclonal antibodies, multispecific antibodies (e.g., bispecific antibodies), nanobodies, monobodies, and antibody fragments so long as they exhibit the desired antigen-binding activity. Other examples of antibodies include, without limitation, a dual-variable immunoglobulin domain, a single-chain Fv molecule (scFv), a single domain antibody (sdAb; e.g., a heavy chain only antibody), a diabody, a triabody, an antibody-like protein scaffold, a Fv fragment, a Fab fragment, a F(ab′)₂ molecule, and a tandem di-scFv.

Further, the term “antibody” includes an immunoglobulin molecule comprising, one or more heavy (H) chains and/or one or more light (L) chains. The chains may be inter-connected by disulfide bonds, as well as multimers thereof (e.g., IgM). Each heavy chain (HC) comprises a heavy chain variable region (or domain) (abbreviated herein as HCVR or VH) and a heavy chain constant region (or domain). The heavy chain constant region comprises three domains, CH1, CH2 and CH3. Each light chain (LC) comprises a light chain variable region (abbreviated herein as LCVR or VL) and a light chain constant region. The light chain constant region comprises one domain (CL1). Each VH and VL is composed of three CDRs and four FRs, arranged from amino-terminus to carboxy-terminus in the following order: FR1, CDR1, FR2, CDR2, 1-R3, CDR3, FR4 Immunoglobulin molecules can be of any type (e.g., IgG, IgE, IgM, IgD, IgA and IgY), class (e.g., IgG1, IgG2, IgG3, IgG4, IgA1 and IgA2) or subclass. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

An “intact” or a “full length” antibody, as used herein, refers to an antibody comprising four polypeptide chains, two heavy (H) chains and two light (L) chains. In one embodiment, an intact antibody is an intact IgG antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “human antibody”, as used herein, refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of one mammalian species, such as a mouse, have been grafted onto human framework sequences. Additional framework region modifications may be made within the human framework sequences. A “humanized form” of an antibody, e.g., a non-human antibody, refers to an antibody that has undergone humanization.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

An “antibody fragment”, “antigen-binding fragment” or “antigen-binding portion” of an antibody refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv); single domain antibodies (sdAbs), and multispecific antibodies formed from antibody fragments.

As used herein, the term “specifically binds” refers to the ability of a binding protein (e.g., a full-length, antibody or antigen-binding fragment, such as a scFv or sdAb) to recognize and form a complex with a target molecule (e.g., CD3) rather than to other proteins, and that is relatively stable under physiologic conditions. Specific binding can be characterized by an equilibrium dissociation constant of at least about 1×10⁶ M or less (e.g., a smaller equilibrium dissociation constant denotes tighter binding). Methods for determining whether two molecules specifically bind are well known in the art and include, for example, equilibrium dialysis, surface plasmon resonance, and the like.

As used herein, the term “does not detectably bind” refers to an antibody that does not bind a cell (e.g., a genetically-modified cell) at a level significantly greater than background, e.g., binds to the cell at a level less than 10%, 8%, 6%, 5%, or 1% above background. In some embodiments, the antibody binds to the cell at a level less than 10%, 8%, 6%, 5%, or 1% more than an isotype control antibody. In one example, the binding is detected by Western blotting, flow cytometry, ELISA, antibody panning, and/or Biacore analysis.

As used herein, “detectable cell-surface expression of CD3” refers to the ability to detect CD3 on the cell surface of a cell (e.g., a genetically-modified cell described herein) using standard experimental methods. Such methods can include, for example, immunostaining and/or flow cytometry specific for CD3. Methods for detecting cell-surface expression of CD3 on a cell include those described in the examples herein, and, for example, those described in MacLeod et al. (2017) Molecular Therapy 25(4): 949-961.

As used herein, “detectable cell-surface expression of an endogenous TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell-surface TCR complex, such as CD3. Methods for detecting cell-surface expression of an endogenous TCR (e.g., an alpha/beta TCR) on an immune cell include those described in the examples herein, and, for example, those described in MacLeod et al. (2017) Molecular Therapy 25(4): 949-961.

As used herein, the term “no detectable CD3 on the cell surface” refers to lack of detection of CD3 on the surface of a genetically-modified cell or population of genetically-modified cells as detected using standard methods in the art (e.g., immunostaining, flow cytometry, ELISA, antibody panning, and/or Biacore analysis). In some embodiments, the genetically-modified cell or population of genetically-modified cells has less than 10%, 8%, 6%, 5%, or 1% of the level of CD3 compared to a corresponding control cell or control cell population.

As used herein, the term “immune cell” refers to any cell that is part of the immune system (innate and/or adaptive) and is of hematopoietic origin. Non-limiting examples of immune cells include lymphocytes, B cells, T cells, monocytes, macrophages, dendritic cells, granulocytes, megakaryocytes, monocytes, macrophages, natural killer cells, myeloid-derived suppressor cells, innate lymphoid cells, platelets, red blood cells, thymocytes, leukocytes, neutrophils, mast cells, eosinophils, basophils, and granulocytes.

As used herein, a “human T cell” or “T cell” refers to a T cell isolated from a donor, particularly a human donor. T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, a “human NK cell” or “NK cell” refers to a NK cell (i.e., a natural killer cell) isolated from a donor, particularly a human donor. NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, a “human B cell” or “B cell” refers to a B cell isolated from a donor, particularly a human donor. B cells, and cells derived therefrom, include isolated T cells that have not been passaged in culture, B cells that have been passaged and maintained under cell culture conditions without immortalization, and B cells that have been immortalized and can be maintained under cell culture conditions indefinitely.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the term “lymphocyte” includes natural killer (NK) cells, T cells, or B cells.

As used herein, the term “deplete a population of lymphocytes” or “lymphodepletion” refers to a reduction of endogenous lymphocytes in a subject, e.g., a reduction of one or more lymphocytes (e.g., NK cells, T cells, and/or B cells) by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject undergoing treatment, relative to a pre-determined threshold, or relative to an untreated subject).

As used herein, the terms “treatment” or “treating a subject” refers to the administration of a genetically-modified cell (e.g., genetically-modified T cell) or population of genetically-modified cells (e.g., a population of genetically-modified T cells) of the invention to a subject having a disease. These terms may also refer to the administration of a lymphodepletion regimen comprising, for example, an anti-CD3 antibody, or antigen-binding fragment thereof. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some aspects, a genetically-modified cell or population of genetically-modified cells described herein is administered during treatment in the form of a pharmaceutical composition of the invention.

The term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In specific embodiments, an effective amount of a genetically-modified cell or population of genetically-modified cells of the invention, or pharmaceutical compositions disclosed herein, reduces at least one symptom of a disease in a subject. In those embodiments wherein the disease is a cancer, an effective amount of the pharmaceutical compositions disclosed herein reduces the level of proliferation or metastasis of cancer, causes a partial or full response or remission of cancer, or reduces at least one symptom of cancer in a subject.

As used herein, the term “effective dose” refers to a dose of a compound administered as part of a lymphodepletion regimen that is sufficient to reduce or eliminate the number of endogenous lymphocytes.

As used herein, the term “cancer” should be understood to encompass any neoplastic disease (whether invasive or metastatic) which is characterized by abnormal and uncontrolled cell division causing malignant growth or tumor.

As used herein, the term “carcinoma” refers to a malignant growth made up of epithelial cells.

As used herein, the term “leukemia” refers to malignancies of the hematopoietic organs/systems and is generally characterized by an abnormal proliferation and development of leukocytes and their precursors in the blood and bone marrow.

As used herein, the term “sarcoma” refers to a tumor which is made up of a substance like the embryonic connective tissue and is generally composed of closely packed cells embedded in a fibrillary, heterogeneous, or homogeneous substance.

As used herein, the term “melanoma” refers to a tumor arising from the melanocytic system of the skin and other organs.

As used herein, the term “lymphoma” refers to a group of blood cell tumors that develop from lymphocytes.

As used herein, the term “blastoma” refers to a type of cancer that is caused by malignancies in precursor cells or blasts (immature or embryonic tissue).

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

Provided herein are methods and compositions for lymphodepletion in a subject in need thereof prior to or during treatment with genetically-modified cells that are modified to lack CD3 on the cell surface, and that express one or more polypeptides of interest (e.g., a chimeric antigen receptor (CAR) or an exogenous T cell receptor (TCR)). Accordingly, further provided herein are compositions and methods for the treatment of a disease, such as cancer, with the genetically-modified cells disclosed herein in a subject who has undergone the lymphodepletion regimens of the invention.

The present invention is based, in part, on the discovery that administration of a biologic (e.g., an antibody) that specifically binds an antigen (e.g., CD3) that is not present on the cell surface of a cellular immunotherapy (e.g., a genetically-modified T cell described herein), but which is expressed on host lymphocytes, aids in the depletion of host immune cells while leaving the cellular immunotherapy unaffected. The present compositions and methods for lymphodepletion are useful to decrease the likelihood or severity of host vs. graft rejection of an immunotherapy, such as an allogeneic cellular immunotherapy, while also allowing for expansion of the incoming cellular immunotherapeutic.

Accordingly, disclosed herein are methods of administering a population of genetically-modified cells having no detectable CD3 on the cell surface and comprising an exogenous polynucleotide encoding a CAR or an exogenous TCR, in order to treat or reduce the symptoms or severity of a disease (e.g., cancer), wherein the individual previously, concomitantly, or subsequently receives treatment with an anti-CD3 antibody (or antigen-binding fragment thereof) and/or a lymphodepleting chemotherapeutic agent in an amount effective to deplete lymphocytes in the subject. In some embodiments, administration of a genetically-modified cell comprising the chimeric antigen receptors or the exogenous TCR disclosed herein, in combination with the lymphodepletion regimens of the invention, treats or reduces the symptoms or severity of diseases, such as cancer, which can be targeted by host cells or genetically-modified cells of the present disclosure. Also disclosed herein are methods of immunotherapy for treating cancer in a subject in need thereof comprising administering to the subject a pharmaceutical composition comprising a host cell or a genetically-modified cell disclosed herein and a pharmaceutically acceptable carrier.

### 2.2 Anti-CD3 Antibodies

The present invention further includes methods of treating a subject with an antibody or antigen binding fragment thereof that specifically binds CD3 (i.e., an anti-CD3 antibody). The anti-CD3 antibodies provided herein are useful in methods for killing CD3+ cells to aid in lymphodepletion before, during, or after administration of the genetically-modified cells provided herein. As the genetically-modified cells of the present invention do not have detectable CD3 on the cell surface, an anti-CD3 antibody can aid in the depletion of endogenous host lymphocytes while leaving the genetically-modified cells unaffected, thereby decreasing the likelihood of host versus graft rejection and promoting cellular expansion of the genetically-modified cells administered to the subject.

Anti-CD3 antibodies include antibodies, and antigen-binding fragments thereof, that specifically bind to a CD3 polypeptide, e.g., a human CD3 polypeptide. The CD3 T cell co-receptor consists of a protein complex comprising a CD3 gamma chain, a CD3 delta chain, and two CD3 epsilon chains. The anti-CD3 antibody can bind an epitope on any domain or region on a CD3 polypeptide. Accordingly, in some embodiments, the anti-CD3 antibodies herein may specifically bind CD3 delta, CD3 epsilon, and/or CD3 gamma.

The anti-CD3 antibody can be any antibody that specifically binds CD3. The CD3 antibody may be of animal (e.g., rodent) or human origin or be a partially or fully humanized antibody. A number of anti-CD3 antibodies are known, including but not limited to muromonab-CD3 (Orthoclone OKT3™), otelixizumab, teplizumab, foralumab (see, e.g., WO2018044948), Resimmune® (Angimmune LLC), or visilizumab. In some embodiments, the antibody is at least 70%, at least 75%, at least 80%, at least 85%, at least 90%, at least 95% homologous with one or more of muromonab-CD3, otelixizumab, teplizumab, foralumab, Resimmune®, or visilizumab.

Some formulations, doses, and dosing schedules for anti-CD3 antibodies, and antigen-binding fragments thereof, are known in the art and are useful in the methods of the invention. For example, some formulations and dosing schedules of muromonab-CD3 (Orthoclone OKT3) can be found, for example, in protocols provided at clinicaltrials.gov under identifiers NCT01287195, NCT01205087, NCT00619372, NCT00027443, NCT00450983, and NCT00004482, and for example in Wilde and Goa, Drugs (1996) Vol. 51(5): 865-894.

Some formulations, doses, and dosing schedules of otelixizumab useful in the methods of the invention can be found, for example, in protocols provided at clinicaltrials.gov under identifiers NCT01222078, NCT02000817, NCT00946257, NCT01077531, NCT01114503, NCT00451321, NCT01123083, and NCT00678886, and for example in Aronson et al., Diabetes Care (2014) Vol. 37(10): 2746-2754.

Some formulations, doses, and dosing schedules of teplizumab useful in the methods of the invention can be found, for example, in protocols provided at clinicaltrials.gov under identifiers NCT04270942, NCT00954915, NCT01030861, NCT03875729, NCT03751007, NCT00378508, NCT01189422, NCT00920582, NCT00870818, and NCT00385697.

Some formulations, doses, and dosing schedules of foralumab useful in the methods of the invention can be found, for example, in protocols provided at clinicaltrials.gov under identifier NCT03291249 and in International Patent Publication Nos. WO2005118635 and WO2018044948.

Some formulations, doses, and dosing schedules of Resimmune® useful in the methods of the invention can be found, for example, in protocols provided at clinicaltrials.gov under identifiers NCT02943642, NCT00611208, NCT02990416, and NCT01888081 and in International Patent Publication No. WO2013158256 and U.S. Pat. Nos. 7,696,338 and 8,217,158.

Some formulations, doses, and dosing schedules of visilizumab useful in the methods of the invention can be found, for example, in protocols provided at clinicaltrials.gov under identifiers NCT00307827, NCT00267306, NCT00279435, NCT00279422, NCT00267709, NCT00267722, NCT00355901, NCT00720629, NCT00502294, NCT00032292, NCT00032279, NCT00032305, and NCT00006009.

In an exemplary embodiment, the antibody, or antigen-binding fragment thereof, that specifically binds to a CD3 polypeptide comprises a heavy chain variable region and a light chain variable region. In one embodiment, the anti-CD3 antibody comprises a heavy chain of an anti-CD3 antibody described herein and a light chain variable region of anti-CD3 antibody described herein. In one embodiment, the anti-CD3 antibody comprises a heavy chain comprising a CDR1, CDR2 and CDR3 of an anti-CD3 antibody described herein, and a light chain variable region comprising a CDR1, CDR2 and CDR3 of an anti-CD3 antibody described herein. In another embodiment, the anti-CD3 antibody is an IgG antibody. In another embodiment, the anti-CD3 antibody is an IgG1 antibody. In another embodiment, the anti-CD3 antibody is an IgG2a antibody. In another embodiment, the anti-CD3 antibody comprises a heavy chain Fc region, which does not bind an Fc receptor or complement.

In another embodiment, the antibody, or antigen-binding fragment thereof, comprises a heavy chain variable region that comprises an amino acid sequence having at least 95% identity to an anti-CD3 antibody herein, e.g., at least 95%, 96%, 97%, 98%, 99%, or 100% identity to an anti-CD3 antibody herein. In certain embodiments, an antibody comprises a modified heavy chain (HC) variable region comprising an HC variable domain of an anti-CD3 antibody herein, or a variant thereof, which variant (i) differs from the anti-CD3 antibody in 1, 2, 3, 4 or 5 amino acids substitutions, additions or deletions; (ii) differs from the anti-CD3 antibody in at most 5, 4, 3, 2, or 1 amino acids substitutions, additions or deletions; (iii) differs from the anti-CD3 antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-CD3 antibody, wherein in any of (i)-(iv), an amino acid substitution may be a conservative amino acid substitution or a non-conservative amino acid substitution.

### 2.3 Additional Lymphodepletion Agents

The present invention further includes compositions including an additional lymphodepletion agent (e.g., a chemotherapeutic agent for lymphodepletion) and methods of treating a subject with the additional lymphodepletion agent prior to administration of the genetically-modified cells provided herein. Pre-treatment or pre-conditioning patients prior to cell therapies with an additional lymphodepletion agent (e.g., a chemotherapeutic agent for lymphodepletion, such as cyclophosphamide and/or fludarabine) improves the efficacy of the cellular therapy by reducing the number of endogenous host lymphocytes in the subject, thereby providing a more optimal environment for administered cells to proliferate once administered to the subject. In some embodiments, 1, 2, 3, 4, or more additional lymphodepletion agents may be combined with the anti-CD3 antibody according to the methods described herein.

A number of additional lymphodepletion agents can be used with the anti-CD3 antibody according to the present methods. In some embodiments, the additional lymphodepletion agent is lymphodepleting but non-myeloablative. Exemplary and non-limiting additional lymphodepletion agents suitable for use with an anti-CD3 antibody include lymphodepleting chemotherapeutic agents such as cyclophosphamide, bendamustine, fludarabine, melphalan, 6-mercaptopurine (6-MP), daunorubicin, cytarabine, L-asparaginase, methotrexate, prednisone, dexamethasone, nelarabine, and additional lymphodepleting antibodies such as anti-CD52 antibodies (e.g., CAMPATH) and rituximab and combinations thereof.

In some embodiments, the additional lymphodepleting agent is a lymphodepleting antibody and/or a lymphodepleting chemotherapeutic agent. In some embodiments, the additional lymphodepleting agent is a lymphodepleting chemotherapeutic agent. In some embodiments, the additional lymphodepleting agent is a lymphodepleting antibody. In some embodiments, the lymphodepleting chemotherapeutic agent is cyclophosphamide or fludarabine. In some embodiments, the lymphodepleting chemotherapeutic agent is fludarabine. In some embodiments, the lymphodepleting chemotherapeutic agent is cyclophosphamide. In certain embodiments, the methods herein involve administering a combination of lymphodepleting chemotherapeutic agents, such as a combination of fludarabine and cyclophosphamide. In some embodiments, a subject is treated with a combination of an anti-CD3 antibody or an antigen binding fragment thereof, fludarabine, and cyclophosphamide according to the dosing schedules described herein.

### 2.4 Methods of Lymphodepletion

The present disclosure provides methods of depletion of population of lymphocytes using a lymphodepleting chemotherapeutic agent and/or an antibody that specifically binds CD3 (i.e., an anti-CD3 antibody or antigen binding fragment thereof) prior to or during administration of the genetically-modified cells provided herein (e.g., cells modified to express a chimeric antigen receptor (CAR) or an exogenous T cell receptor (TCR) and modified to lack CD3 or an endogenous TCR on the cell surface). The lymphodepletion methods of the invention are useful to reduce the likelihood or severity of host vs graft rejection of the genetically-modified cells, while also allowing for expansion of the incoming cells.

The additional lymphodepleting agent (e.g., a lymphodepleting chemotherapeutic agent) and/or anti-CD3 antibody, or antigen binding fragment thereof, can be administered in an amount effective to deplete or reduce the quantity of lymphocytes in the subject, for example, by 1%, 5%, 10%, 20%, 30%, 40%, 50%, 60%, 70%, 80%, 90%, 91%, 92%, 93%, 94%, 95%, 96%, 97%, 98%, 99%, or more, relative to a control (e.g., relative to a starting amount in the subject undergoing treatment, relative to a pre-determined threshold, or relative to an untreated subject) prior to administration of the genetically-modified cells. The reduction in lymphocyte count can be monitored using conventional techniques known in the art, such as by flow cytometry analysis of cells expressing characteristic lymphocyte cell surface antigens in a blood sample withdrawn from the subject at varying intervals during treatment with the antibody. According to some embodiments, when the concentration of lymphocytes has reached a minimum value in response to lymphodepletion therapy with an anti-CD3 antibody or antigen binding fragment thereof and/or additional lymphodepletion agent (e.g., a lymphodepleting chemotherapeutic agent), the physician may conclude the lymphodepletion therapy and may begin preparing the subject for administration of the genetically-modified cells provided herein. In certain embodiments, the anti-CD3 antibody can alternatively or additionally be administered one or more times during or following the initial administration of the genetically-modified cells as the genetically-modified cells provided herein do not have detectable levels of CD3 on the cell surface.

In some embodiments, the anti-CD3 antibody or antigen-binding fragment thereof can be administered to a subject at a dosage that is suitable for reducing CD3+ lymphocytes in the subject. In some embodiments, the dosage of anti-CD3 antibody is from about 0.001 mg/kg to about 150 mg/kg. In some embodiments, the dosage of anti-CD3 antibody is from about 0.001 mg/kg to about 50 mg/kg. In some embodiments, the dosage of anti-CD3 antibody is from about 0.001 mg/kg to about 15 mg/kg. In some embodiments, the dosage of anti-CD3 antibody is from about 0.001 mg/kg to about 10 mg/kg. In some embodiments, the dosage of anti-CD3 antibody is from about 0.001 mg/kg to about 5 mg/kg. In some embodiments, the dosage of anti-CD3 antibody is from about 0.001 mg/kg to about 2.5 mg/kg. In some embodiments, the dosage of anti-CD3 antibody is from about 0.001 mg/kg to about 1.0 mg/kg. In some embodiments, the dosage of anti-CD3 antibody is from about 0.01 mg/kg to about 1.0 mg/kg. In some embodiments, the dosage of anti-CD3 antibody is from about 0.001 mg/kg to about 0.5 mg/kg. In some embodiments, the dosage of anti-CD3 antibody is from about 0.01 mg/kg to about 0.5 mg/kg. In some embodiments, the dosage of anti-CD3 antibody is from about 0.01 mg/kg to about 0.05 mg/kg. In some embodiments, the dosage of anti-CD3 antibody is about 0.001 mg/kg, about 0.005, about 0.01, about 0.05 mg/kg, about 0.1, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, or about 150 mg/kg. In some embodiments, the dosage of anti-CD3 antibody is about 0.001 mg/kg. In some embodiments, the dosage of anti-CD3 antibody is about 0.005 mg/kg. In some embodiments, the dosage of anti-CD3 antibody is about 0.01 mg/kg. In some embodiments, the dosage of anti-CD3 antibody is about 0.05 mg/kg. In some embodiments, the dosage of anti-CD3 antibody is about 0.1 mg/kg. In some embodiments, the dosage of anti-CD3 antibody is about 0.5 mg/kg. In some embodiments, the dosage of anti-CD3 antibody is about 1 mg/kg. In some embodiments, the dosage of anti-CD3 antibody is about 2.5 mg/kg. In some embodiments, the dosage of anti-CD3 antibody is about 5 mg/kg. In some embodiments, the dosage of anti-CD3 antibody is about 10 mg/kg. In some embodiments, the dosage of anti-CD3 antibody is about 15 mg/kg. In some embodiments, the dosage of anti-CD3 antibody is about 20 mg/kg. In some embodiments, the dosage of anti-CD3 antibody is about 25 mg/kg. In some embodiments, the dosage of anti-CD3 antibody is about 30 mg/kg. In some embodiments, the dosage of anti-CD3 antibody is about 35 mg/kg. In some embodiments, the dosage of anti-CD3 antibody is about 40 mg/kg. In some embodiments, the dosage of anti-CD3 antibody is about 45 mg/kg. In some embodiments, the dosage of anti-CD3 antibody is about 50 mg/kg. In some embodiments, the dosage of anti-CD3 antibody is about 60 mg/kg. In some embodiments, the dosage of anti-CD3 antibody is about 70 mg/kg. In some embodiments, the dosage of anti-CD3 antibody is about 80 mg/kg. In some embodiments, the dosage of anti-CD3 antibody is about 90 mg/kg. In some embodiments, the dosage of anti-CD3 antibody is about 100 mg/kg. In some embodiments, the dosage of anti-CD3 antibody is about 110 mg/kg. In some embodiments, the dosage of anti-CD3 antibody is about 120 mg/kg. In some embodiments, the dosage of anti-CD3 antibody is about 130 mg/kg. In some embodiments, the dosage of anti-CD3 antibody is about 140 mg/kg. In some embodiments, the dosage of anti-CD3 antibody is about 150 mg/kg. In some embodiments, the dosage of anti-CD3 antibody is about 0.1 mg/day. In some embodiments, the dosage of anti-CD3 antibody is about 0.25 mg/day. In some embodiments, the dosage of anti-CD3 antibody is about 0.5 mg/day. In some embodiments, the dosage of anti-CD3 antibody is about 0.75 mg/day. In some embodiments, the dosage of anti-CD3 antibody is about 1 mg/day. In some embodiments, the dosage of anti-CD3 antibody is about 2.5 mg/day. In some embodiments, the dosage of anti-CD3 antibody is about 5 mg/day. In some embodiments, the dosage of anti-CD3 antibody is about 7.5 mg/day. In some embodiments, the dosage of anti-CD3 antibody is about 10 mg/day. In some embodiments, the dosage of anti-CD3 antibody is about 15 mg/day. In some embodiments, the dosage of anti-CD3 antibody is about 20 mg/day. In some embodiments, the dosage of anti-CD3 antibody is about 25 mg/day. In some embodiments, the dosage of anti-CD3 antibody is about 30 mg/day. In some embodiments, the dosage of anti-CD3 antibody is about 35 mg/day. In some embodiments, the dosage of anti-CD3 antibody is about 40 mg/day. In some embodiments, the dosage of anti-CD3 antibody is about 45 mg/day. In some embodiments, the dosage of anti-CD3 antibody is about 50 mg/day. In some embodiments, the dosage of anti-CD3 antibody is about 75 mg/day. In some embodiments, the dosage of anti-CD3 antibody is about 100 mg/day. In some embodiments, the dosage of anti-CD3 antibody is about 200 mg/day. In some embodiments, the dosage of anti-CD3 antibody is about 300 mg/day. In some embodiments, the dosage of anti-CD3 antibody is about 400 mg/day. In some embodiments, the dosage of anti-CD3 antibody is about 500 mg/day. In some embodiments, the dosage of anti-CD3 antibody is about 600 mg/day. In some embodiments, the dosage of anti-CD3 antibody is about 700 mg/day. In some embodiments, the dosage of anti-CD3 antibody is about 800 mg/day. In some embodiments, the dosage of anti-CD3 antibody is about 900 mg/day. In some embodiments, the dosage of anti-CD3 antibody is about 1000 mg/day. In some embodiments, the dosage of anti-CD3 antibody is about 2000 mg/day. In some embodiments, the dosage of anti-CD3 antibody is about 3000 mg/day. In some embodiments, the dosage of anti-CD3 antibody is about 4000 mg/day. In some embodiments, the dosage of anti-CD3 antibody is about 5000 mg/day. In some embodiments, the dosage of anti-CD3 antibody is about 6000 mg/day. In some embodiments, the dosage of anti-CD3 antibody is about 7000 mg/day. In some embodiments, the dosage of anti-CD3 antibody is about 8000 mg/day. In some embodiments, the dosage of anti-CD3 antibody is about 9000 mg/day. In some embodiments, the dosage of anti-CD3 antibody is about 10000 mg/day. In some embodiments, the anti-CD3 antibody is administered prior to, during, or following administration of a genetically-modified cell to the subject.

The anti-CD3 antibody or antigen-binding fragment thereof can be administered to the subject at a time that is effective to deplete lymphocytes in the subject. For instance, in some embodiments, the anti-CD3 antibody or antigen-binding fragment thereof is administered to the subject from 1 hour to 1 month or more prior to administration of the genetically modified cells. Thus, in some embodiments, the anti-CD3 antibody is administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more prior to administration of the genetically-modified cells.

In some embodiments, the anti-CD3 antibody is administered daily starting 14 days prior to administration of the genetically-modified cells and ending 1 day prior to administration of the genetically-modified cells. In some embodiments, the anti-CD3 antibody is administered daily starting 13 days prior to administration of the genetically-modified cells and ending 1 day prior to administration of the genetically-modified cells. In some embodiments, the anti-CD3 antibody is administered daily starting 12 days prior to administration of the genetically-modified cells and ending 1 day prior to administration of the genetically-modified cells. In some embodiments, the anti-CD3 antibody is administered daily starting 11 days prior to administration of the genetically-modified cells and ending 1 day prior to administration of the genetically-modified cells. In some embodiments, the anti-CD3 antibody is administered daily starting 10 days prior to administration of the genetically-modified cells and ending 1 day prior to administration of the genetically-modified cells. In some embodiments, the anti-CD3 antibody is administered daily starting 9 days prior to administration of the genetically-modified cells and ending 1 day prior to administration of the genetically-modified cells. In some embodiments, the anti-CD3 antibody is administered daily starting 8 days prior to administration of the genetically-modified cells and ending 1 day prior to administration of the genetically-modified cells. In some embodiments, the anti-CD3 antibody is administered daily starting 7 days prior to administration of the genetically-modified cells and ending 1 day prior to administration of the genetically-modified cells. In some embodiments, the anti-CD3 antibody is administered daily starting 6 days prior to administration of the genetically-modified cells and ending 1 day prior to administration of the genetically-modified cells. In some embodiments, the anti-CD3 antibody is administered daily starting 5 days prior to administration of the genetically-modified cells and ending 1 day prior to administration of the genetically-modified cells. In some embodiments, the anti-CD3 antibody is administered daily starting 4 days prior to administration of the genetically-modified cells and ending 1 day prior to administration of the genetically-modified cells. In some embodiments, the anti-CD3 antibody is administered daily starting 3 days prior to administration of the genetically-modified cells and ending 1 day prior to administration of the genetically-modified cells. In some embodiments, the anti-CD3 antibody is administered daily starting 2 days prior to administration of the genetically-modified cells and ending 1 day prior to administration of the genetically-modified cells. In some embodiments, the anti-CD3 antibody is administered 1 day prior to administration of the genetically-modified cells.

In some embodiments, the anti-CD3 antibody is administered daily starting 14 days prior to administration of the genetically-modified cells and ending 2 days prior to administration of the genetically-modified cells. In some embodiments, the anti-CD3 antibody is administered daily starting 13 days prior to administration of the genetically-modified cells and ending 2 days prior to administration of the genetically-modified cells. In some embodiments, the anti-CD3 antibody is administered daily starting 12 days prior to administration of the genetically-modified cells and ending 2 days prior to administration of the genetically-modified cells. In some embodiments, the anti-CD3 antibody is administered daily starting 11 days prior to administration of the genetically-modified cells and ending 2 days prior to administration of the genetically-modified cells. In some embodiments, the anti-CD3 antibody is administered daily starting 10 days prior to administration of the genetically-modified cells and ending 2 days prior to administration of the genetically-modified cells. In some embodiments, the anti-CD3 antibody is administered daily starting 9 days prior to administration of the genetically-modified cells and ending 2 days prior to administration of the genetically-modified cells. In some embodiments, the anti-CD3 antibody is administered daily starting 8 days prior to administration of the genetically-modified cells and ending 2 days prior to administration of the genetically-modified cells. In some embodiments, the anti-CD3 antibody is administered daily starting 7 days prior to administration of the genetically-modified cells and ending 2 days prior to administration of the genetically-modified cells. In some embodiments, the anti-CD3 antibody is administered daily starting 6 days prior to administration of the genetically-modified cells and ending 2 days prior to administration of the genetically-modified cells. In some embodiments, the anti-CD3 antibody is administered daily starting 5 days prior to administration of the genetically-modified cells and ending 2 days prior to administration of the genetically-modified cells. In some embodiments, the anti-CD3 antibody is administered daily starting 4 days prior to administration of the genetically-modified cells and ending 2 days prior to administration of the genetically-modified cells. In some embodiments, the anti-CD3 antibody is administered daily starting 3 days prior to administration of the genetically-modified cells and ending 2 days prior to administration of the genetically-modified cells. In some embodiments, the anti-CD3 antibody is administered 2 days prior to administration of the genetically-modified cells.

In some embodiments, the anti-CD3 antibody is administered daily starting 14 days prior to administration of the genetically-modified cells and ending 3 days prior to administration of the genetically-modified cells. In some embodiments, the anti-CD3 antibody is administered daily starting 13 days prior to administration of the genetically-modified cells and ending 3 days prior to administration of the genetically-modified cells. In some embodiments, the anti-CD3 antibody is administered daily starting 12 days prior to administration of the genetically-modified cells and ending 3 days prior to administration of the genetically-modified cells. In some embodiments, the anti-CD3 antibody is administered daily starting 11 days prior to administration of the genetically-modified cells and ending 3 days prior to administration of the genetically-modified cells. In some embodiments, the anti-CD3 antibody is administered daily starting 10 days prior to administration of the genetically-modified cells and ending 3 days prior to administration of the genetically-modified cells. In some embodiments, the anti-CD3 antibody is administered daily starting 9 days prior to administration of the genetically-modified cells and ending 3 days prior to administration of the genetically-modified cells. In some embodiments, the anti-CD3 antibody is administered daily starting 8 days prior to administration of the genetically-modified cells and ending 3 days prior to administration of the genetically-modified cells. In some embodiments, the anti-CD3 antibody is administered daily starting 7 days prior to administration of the genetically-modified cells and ending 3 days prior to administration of the genetically-modified cells. In some embodiments, the anti-CD3 antibody is administered daily starting 6 days prior to administration of the genetically-modified cells and ending 3 days prior to administration of the genetically-modified cells. In some embodiments, the anti-CD3 antibody is administered daily starting 5 days prior to administration of the genetically-modified cells and ending 3 days prior to administration of the genetically-modified cells. In some embodiments, the anti-CD3 antibody is administered daily starting 4 days prior to administration of the genetically-modified cells and ending 3 days prior to administration of the genetically-modified cells. In some embodiments, the anti-CD3 antibody is administered 3 days prior to administration of the genetically-modified cells.

In some embodiments, the anti-CD3 antibody is administered daily starting 14 days prior to administration of the genetically-modified cells and ending 4 days prior to administration of the genetically-modified cells. In some embodiments, the anti-CD3 antibody is administered daily starting 13 days prior to administration of the genetically-modified cells and ending 4 days prior to administration of the genetically-modified cells. In some embodiments, the anti-CD3 antibody is administered daily starting 12 days prior to administration of the genetically-modified cells and ending 4 days prior to administration of the genetically-modified cells. In some embodiments, the anti-CD3 antibody is administered daily starting 11 days prior to administration of the genetically-modified cells and ending 4 days prior to administration of the genetically-modified cells. In some embodiments, the anti-CD3 antibody is administered daily starting 10 days prior to administration of the genetically-modified cells and ending 4 days prior to administration of the genetically-modified cells. In some embodiments, the anti-CD3 antibody is administered daily starting 9 days prior to administration of the genetically-modified cells and ending 4 days prior to administration of the genetically-modified cells. In some embodiments, the anti-CD3 antibody is administered daily starting 8 days prior to administration of the genetically-modified cells and ending 4 days prior to administration of the genetically-modified cells. In some embodiments, the anti-CD3 antibody is administered daily starting 7 days prior to administration of the genetically-modified cells and ending 4 days prior to administration of the genetically-modified cells. In some embodiments, the anti-CD3 antibody is administered daily starting 6 days prior to administration of the genetically-modified cells and ending 4 days prior to administration of the genetically-modified cells. In some embodiments, the anti-CD3 antibody is administered daily starting 5 days prior to administration of the genetically-modified cells and ending 4 days prior to administration of the genetically-modified cells. In some embodiments, the anti-CD3 antibody is administered 4 days prior to administration of the genetically-modified cells.

In some embodiments, the anti-CD3 antibody is administered every other day starting 13 days prior to administration of the genetically-modified cells and ending 1 day prior to administration of the genetically-modified cells. In some embodiments, the anti-CD3 antibody is administered every other day starting 11 days prior to administration of the genetically-modified cells and ending 1 day prior to administration of the genetically-modified cells. In some embodiments, the anti-CD3 antibody is administered every other day starting 9 days prior to administration of the genetically-modified cells and ending 1 day prior to administration of the genetically-modified cells. In some embodiments, the anti-CD3 antibody is administered every other day starting 7 days prior to administration of the genetically-modified cells and ending 1 day prior to administration of the genetically-modified cells. In some embodiments, the anti-CD3 antibody is administered every other day starting 5 days prior to administration of the genetically-modified cells and ending 1 day prior to administration of the genetically-modified cells. In some embodiments, the anti-CD3 antibody is administered every other day starting 3 days prior to administration of the genetically-modified cells and ending 1 day prior to administration of the genetically-modified cells.

In some embodiments, the anti-CD3 antibody is administered every other day starting 13 days prior to administration of the genetically-modified cells and ending 3 days prior to administration of the genetically-modified cells. In some embodiments, the anti-CD3 antibody is administered every other day starting 11 days prior to administration of the genetically-modified cells and ending 3 days prior to administration of the genetically-modified cells. In some embodiments, the anti-CD3 antibody is administered every other day starting 9 days prior to administration of the genetically-modified cells and ending 3 days prior to administration of the genetically-modified cells. In some embodiments, the anti-CD3 antibody is administered every other day starting 7 days prior to administration of the genetically-modified cells and ending 3 days prior to administration of the genetically-modified cells. In some embodiments, the anti-CD3 antibody is administered every other day starting 5 days prior to administration of the genetically-modified cells and ending 3 days prior to administration of the genetically-modified cells.

In some embodiments, the anti-CD3 antibody is administered every other day starting 14 days prior to administration of the genetically-modified cells and ending 2 days prior to administration of the genetically-modified cells. In some embodiments, the anti-CD3 antibody is administered every other day starting 12 days prior to administration of the genetically-modified cells and ending 2 days prior to administration of the genetically-modified cells. In some embodiments, the anti-CD3 antibody is administered every other day starting 10 days prior to administration of the genetically-modified cells and ending 2 days prior to administration of the genetically-modified cells. In some embodiments, the anti-CD3 antibody is administered every other day starting 8 days prior to administration of the genetically-modified cells and ending 2 days prior to administration of the genetically-modified cells. In some embodiments, the anti-CD3 antibody is administered every other day starting 6 days prior to administration of the genetically-modified cells and ending 2 days prior to administration of the genetically-modified cells. In some embodiments, the anti-CD3 antibody is administered every other day starting 4 days prior to administration of the genetically-modified cells and ending 2 days prior to administration of the genetically-modified cells.

In some embodiments, the anti-CD3 antibody is administered every other day starting 14 days prior to administration of the genetically-modified cells and ending 4 days prior to administration of the genetically-modified cells. In some embodiments, the anti-CD3 antibody is administered every other day starting 12 days prior to administration of the genetically-modified cells and ending 4 days prior to administration of the genetically-modified cells. In some embodiments, the anti-CD3 antibody is administered every other day starting 10 days prior to administration of the genetically-modified cells and ending 4 days prior to administration of the genetically-modified cells. In some embodiments, the anti-CD3 antibody is administered every other day starting 8 days prior to administration of the genetically-modified cells and ending 4 days prior to administration of the genetically-modified cells. In some embodiments, the anti-CD3 antibody is administered every other day starting 6 days prior to administration of the genetically-modified cells and ending 4 days prior to administration of the genetically-modified cells.

In some embodiments, the anti-CD3 antibody or antigen-binding fragment thereof is administered to the subject concomitant with the administration of the genetically-modified cells.

In some embodiments, the anti-CD3 antibody or antigen-binding fragment thereof is administered to the subject from 1 hour to one month or more following administration of the genetically-modified cells. Accordingly, in some embodiments, the anti-CD3 antibody is administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more following administration of the genetically-modified cells. In some embodiments, the anti-CD3 antibody is administered prior to administration of the genetically modified cells and following administration of the genetically modified cells for any of the preceding described lengths of time.

In some embodiments, the anti-CD3 antibody is dosed prior to the administration of the genetically-modified cells and continued after the administration of the genetically modified cells. In some embodiments, the anti-CD3 antibody is administered 1 hour following administration of the genetically-modified cells. In some embodiments, the anti-CD3 antibody is administered 12 hours following administration of the genetically-modified cells. In some embodiments, the anti-CD3 antibody is administered 1 day following administration of the genetically-modified cells. In some embodiments, the anti-CD3 antibody is administered 2 days following administration of the genetically-modified cells. In some embodiments, the anti-CD3 antibody is administered 3 days following administration of the genetically-modified cells. In some embodiments, the anti-CD3 antibody is administered 4 days following administration of the genetically-modified cells. In some embodiments, the anti-CD3 antibody is administered 5 days following administration of the genetically-modified cells. In some embodiments, the anti-CD3 antibody is administered 6 days following administration of the genetically-modified cells. In some embodiments, the anti-CD3 antibody is administered 7 days following administration of the genetically-modified cells. In some embodiments, the anti-CD3 antibody is administered 8 days following administration of the genetically-modified cells. In some embodiments, the anti-CD3 antibody is administered 9 days following administration of the genetically-modified cells. In some embodiments, the anti-CD3 antibody is administered 10 days following administration of the genetically-modified cells. In some embodiments, the anti-CD3 antibody is administered 11 days following administration of the genetically-modified cells. In some embodiments, the anti-CD3 antibody is administered 12 days following administration of the genetically-modified cells. In some embodiments, the anti-CD3 antibody is administered 13 days following administration of the genetically-modified cells. In some embodiments, the anti-CD3 antibody is administered 14 days following administration of the genetically-modified cells. In some embodiments, the anti-CD3 antibody is administered 15 days following administration of the genetically-modified cells. In some embodiments, the anti-CD3 antibody is administered 16 days following administration of the genetically-modified cells. In some embodiments, the anti-CD3 antibody is administered 17 days following administration of the genetically-modified cells. In some embodiments, the anti-CD3 antibody is administered 18 days following administration of the genetically-modified cells. In some embodiments, the anti-CD3 antibody is administered 19 days following administration of the genetically-modified cells. In some embodiments, the anti-CD3 antibody is administered 20 days following administration of the genetically-modified cells. In some embodiments, the anti-CD3 antibody is administered 21 days following administration of the genetically-modified cells. In some embodiments, the anti-CD3 antibody is administered 22 days following administration of the genetically-modified cells. In some embodiments, the anti-CD3 antibody is administered 23 days following administration of the genetically-modified cells. In some embodiments, the anti-CD3 antibody is administered 24 days following administration of the genetically-modified cells. In some embodiments, the anti-CD3 antibody is administered 25 days following administration of the genetically-modified cells. In some embodiments, the anti-CD3 antibody is administered 26 days following administration of the genetically-modified cells. In some embodiments, the anti-CD3 antibody is administered 27 days following administration of the genetically-modified cells. In some embodiments, the anti-CD3 antibody is administered 28 days following administration of the genetically-modified cells. In some embodiments, the anti-CD3 antibody is administered 29 days following administration of the genetically-modified cells. In some embodiments, the anti-CD3 antibody is administered 30 days following administration of the genetically-modified cells.

In some embodiments, the anti-CD3 antibody is administered daily following administration of the genetically-modified cells. In some embodiments, the anti-CD3 antibody is administered every other day following administration of the genetically-modified cells. In some embodiments, the anti-CD3 antibody is administered every three days following administration of the genetically-modified cells. In some embodiments, the anti-CD3 antibody is administered every four days following administration of the genetically-modified cells. In some embodiments, the anti-CD3 antibody is administered every five days following administration of the genetically-modified cells. In some embodiments, the anti-CD3 antibody is administered every six days following administration of the genetically-modified cells. In some embodiments, the anti-CD3 antibody is administered every seven days following administration of the genetically-modified cells.

In some embodiments, the anti-CD3 antibody is administered on the same day as administration of the genetically-modified cells. In some embodiments, the anti-CD3 antibody is administered daily starting the same day as administration of the genetically-modified cells and ending 1 day after administration of the genetically-modified cells. In some embodiments, the anti-CD3 antibody is administered daily starting the same day as administration of the genetically-modified cells and ending 2 days after administration of the genetically-modified cells. In some embodiments, the anti-CD3 antibody is administered daily starting the same day as administration of the genetically-modified cells and ending 3 days after administration of the genetically-modified cells. In some embodiments, the anti-CD3 antibody is administered daily starting the same day as administration of the genetically-modified cells and ending 4 days after administration of the genetically-modified cells. In some embodiments, the anti-CD3 antibody is administered daily starting the same day as administration of the genetically-modified cells and ending 5 days after administration of the genetically-modified cells. In some embodiments, the anti-CD3 antibody is administered daily starting the same day as administration of the genetically-modified cells and ending 6 days after administration of the genetically-modified cells. In some embodiments, the anti-CD3 antibody is administered daily starting the same day as administration of the genetically-modified cells and ending 7 days after administration of the genetically-modified cells. In some embodiments, the anti-CD3 antibody is administered daily starting the same day as administration of the genetically-modified cells and ending 8 days after administration of the genetically-modified cells. In some embodiments, the anti-CD3 antibody is administered daily starting the same day as administration of the genetically-modified cells and ending 9 days after administration of the genetically-modified cells. In some embodiments, the anti-CD3 antibody is administered daily starting the same day as administration of the genetically-modified cells and ending 10 days after administration of the genetically-modified cells. In some embodiments, the anti-CD3 antibody is administered daily starting the same day as administration of the genetically-modified cells and ending 11 days after administration of the genetically-modified cells. In some embodiments, the anti-CD3 antibody is administered daily starting the same day as administration of the genetically-modified cells and ending 12 days after administration of the genetically-modified cells. In some embodiments, the anti-CD3 antibody is administered daily starting the same day as administration of the genetically-modified cells and ending 13 days after administration of the genetically-modified cells. In some embodiments, the anti-CD3 antibody is administered daily starting the same day as administration of the genetically-modified cells and ending 14 days after administration of the genetically-modified cells. In some embodiments, the anti-CD3 antibody is administered daily starting the same day as administration of the genetically-modified cells and ending more than 14 days after administration of the genetically-modified cells.

In some embodiments, the anti-CD3 antibody is administered daily starting 1 day after administration of the genetically-modified cells and ending 2 days after administration of the genetically-modified cells. In some embodiments, the anti-CD3 antibody is administered daily starting 1 day after administration of the genetically-modified cells and ending 3 days after administration of the genetically-modified cells. In some embodiments, the anti-CD3 antibody is administered daily starting 1 day after administration of the genetically-modified cells and ending 4 days after administration of the genetically-modified cells. In some embodiments, the anti-CD3 antibody is administered daily starting 1 day after administration of the genetically-modified cells and ending 5 days after administration of the genetically-modified cells. In some embodiments, the anti-CD3 antibody is administered daily starting 1 day after administration of the genetically-modified cells and ending 6 days after administration of the genetically-modified cells. In some embodiments, the anti-CD3 antibody is administered daily starting 1 day after administration of the genetically-modified cells and ending 7 days after administration of the genetically-modified cells. In some embodiments, the anti-CD3 antibody is administered daily starting 1 day after administration of the genetically-modified cells and ending 8 days after administration of the genetically-modified cells. In some embodiments, the anti-CD3 antibody is administered daily starting 1 day after administration of the genetically-modified cells and ending 9 days after administration of the genetically-modified cells. In some embodiments, the anti-CD3 antibody is administered daily starting 1 day after administration of the genetically-modified cells and ending 10 days after administration of the genetically-modified cells. In some embodiments, the anti-CD3 antibody is administered daily starting 1 day after administration of the genetically-modified cells and ending 11 days after administration of the genetically-modified cells. In some embodiments, the anti-CD3 antibody is administered daily starting 1 day after administration of the genetically-modified cells and ending 12 days after administration of the genetically-modified cells. In some embodiments, the anti-CD3 antibody is administered daily starting 1 day after administration of the genetically-modified cells and ending 13 days after administration of the genetically-modified cells. In some embodiments, the anti-CD3 antibody is administered daily starting 1 day after administration of the genetically-modified cells and ending 14 days after administration of the genetically-modified cells. In some embodiments, the anti-CD3 antibody is administered daily starting 1 day after administration of the genetically-modified cells and ending more than 14 days after administration of the genetically-modified cells.

In some embodiments, the anti-CD3 antibody is administered daily starting 2 days after administration of the genetically-modified cells and ending 3 days after administration of the genetically-modified cells. In some embodiments, the anti-CD3 antibody is administered daily starting 2 days after administration of the genetically-modified cells and ending 4 days after administration of the genetically-modified cells. In some embodiments, the anti-CD3 antibody is administered daily starting 2 days after administration of the genetically-modified cells and ending 5 days after administration of the genetically-modified cells. In some embodiments, the anti-CD3 antibody is administered daily starting 2 days after administration of the genetically-modified cells and ending 6 days after administration of the genetically-modified cells. In some embodiments, the anti-CD3 antibody is administered daily starting 2 days after administration of the genetically-modified cells and ending 7 days after administration of the genetically-modified cells. In some embodiments, the anti-CD3 antibody is administered daily starting 2 days after administration of the genetically-modified cells and ending 8 days after administration of the genetically-modified cells. In some embodiments, the anti-CD3 antibody is administered daily starting 2 days after administration of the genetically-modified cells and ending 9 days after administration of the genetically-modified cells. In some embodiments, the anti-CD3 antibody is administered daily starting 2 days after administration of the genetically-modified cells and ending 10 days after administration of the genetically-modified cells. In some embodiments, the anti-CD3 antibody is administered daily starting 2 days after administration of the genetically-modified cells and ending 11 days after administration of the genetically-modified cells. In some embodiments, the anti-CD3 antibody is administered daily starting 2 days after administration of the genetically-modified cells and ending 12 days after administration of the genetically-modified cells. In some embodiments, the anti-CD3 antibody is administered daily starting 2 days after administration of the genetically-modified cells and ending 13 days after administration of the genetically-modified cells. In some embodiments, the anti-CD3 antibody is administered daily starting 2 days after administration of the genetically-modified cells and ending 14 days after administration of the genetically-modified cells. In some embodiments, the anti-CD3 antibody is administered daily starting 2 days after administration of the genetically-modified cells and ending more than 14 days after administration of the genetically-modified cells.

In some embodiments, the anti-CD3 antibody is administered every other day starting the same day as administration of the genetically-modified cells and ending 2 days after administration of the genetically-modified cells. In some embodiments, the anti-CD3 antibody is administered every other day starting the same day as administration of the genetically-modified cells and ending 4 days after administration of the genetically-modified cells. In some embodiments, the anti-CD3 antibody is administered every other day starting the same day as administration of the genetically-modified cells and ending 6 days after administration of the genetically-modified cells. In some embodiments, the anti-CD3 antibody is administered every other day starting the same day as administration of the genetically-modified cells and ending 8 days after administration of the genetically-modified cells. In some embodiments, the anti-CD3 antibody is administered every other day starting the same day as administration of the genetically-modified cells and ending 10 days after administration of the genetically-modified cells. In some embodiments, the anti-CD3 antibody is administered every other day starting the same day as administration of the genetically-modified cells and ending 12 days after administration of the genetically-modified cells. In some embodiments, the anti-CD3 antibody is administered every other day starting the same day as administration of the genetically-modified cells and ending 14 days after administration of the genetically-modified cells. In some embodiments, the anti-CD3 antibody is administered every other day starting the same day as administration of the genetically-modified cells and ending more than 14 days after administration of the genetically-modified cells.

In some embodiments, the anti-CD3 antibody is administered every other day starting 1 day after administration of the genetically-modified cells and ending 3 days after administration of the genetically-modified cells. In some embodiments, the anti-CD3 antibody is administered every other day starting 1 day after administration of the genetically-modified cells and ending 5 days after administration of the genetically-modified cells. In some embodiments, the anti-CD3 antibody is administered every other day starting 1 day after administration of the genetically-modified cells and ending 7 days after administration of the genetically-modified cells. In some embodiments, the anti-CD3 antibody is administered every other day starting 1 day after administration of the genetically-modified cells and ending 9 days after administration of the genetically-modified cells. In some embodiments, the anti-CD3 antibody is administered every other day starting 1 day after administration of the genetically-modified cells and ending 11 days after administration of the genetically-modified cells. In some embodiments, the anti-CD3 antibody is administered every other day starting 1 day after administration of the genetically-modified cells and ending 13 days after administration of the genetically-modified cells. In some embodiments, the anti-CD3 antibody is administered every other day starting 1 day after administration of the genetically-modified cells and ending 15 days after administration of the genetically-modified cells. In some embodiments, the anti-CD3 antibody is administered every other day starting 1 day after administration of the genetically-modified cells and ending more than 15 days after administration of the genetically-modified cells.

In some embodiments, the anti-CD3 antibody is administered every other day starting 2 days after administration of the genetically-modified cells and ending 4 days after administration of the genetically-modified cells. In some embodiments, the anti-CD3 antibody is administered every other day starting 2 days after administration of the genetically-modified cells and ending 6 days after administration of the genetically-modified cells. In some embodiments, the anti-CD3 antibody is administered every other day starting 2 days after administration of the genetically-modified cells and ending 8 days after administration of the genetically-modified cells. In some embodiments, the anti-CD3 antibody is administered every other day starting 2 days after administration of the genetically-modified cells and ending 10 days after administration of the genetically-modified cells. In some embodiments, the anti-CD3 antibody is administered every other day starting 2 days after administration of the genetically-modified cells and ending 12 days after administration of the genetically-modified cells. In some embodiments, the anti-CD3 antibody is administered every other day starting 2 days after administration of the genetically-modified cells and ending 14 days after administration of the genetically-modified cells. In some embodiments, the anti-CD3 antibody is administered every other day starting 2 days after administration of the genetically-modified cells and ending more than 14 days after administration of the genetically-modified cells.

In some embodiments, the anti-CD3 antibody is continued to be administered following administration of the genetically-modified cells for a time period necessary to deplete, reduce, or maintain a reduction in the quantity of host lymphocytes in the subject, for example, by 1%, 5%, 10%, 20%, 30%, 40%, 50%, 60%, 70%, 80%, 90%, 91%, 92%, 93%, 94%, 95%, 96%, 97%, 98%, 99%, or more, relative to a control. Accordingly, in some embodiments, the anti-CD3 antibody is administered for 1 day following administration of the genetically-modified cells. In some embodiments, the anti-CD3 antibody is administered for 2 days following administration of the genetically-modified cells. In some embodiments, the anti-CD3 antibody is administered for 3 days following administration of the genetically-modified cells. In some embodiments, the anti-CD3 antibody is administered for 4 days following administration of the genetically-modified cells. In some embodiments, the anti-CD3 antibody is administered for 5 days following administration of the genetically-modified cells. In some embodiments, the anti-CD3 antibody is administered for 6 days following administration of the genetically-modified cells. In some embodiments, the anti-CD3 antibody is administered for 7 days following administration of the genetically-modified cells. In some embodiments, the anti-CD3 antibody is administered for 8 days following administration of the genetically-modified cells. In some embodiments, the anti-CD3 antibody is administered for 9 days following administration of the genetically-modified cells. In some embodiments, the anti-CD3 antibody is administered for 10 days following administration of the genetically-modified cells. In some embodiments, the anti-CD3 antibody is administered for 11 days following administration of the genetically-modified cells. In some embodiments, the anti-CD3 antibody is administered for 12 days following administration of the genetically-modified cells. In some embodiments, the anti-CD3 antibody is administered for 13 days following administration of the genetically-modified cells. In some embodiments, the anti-CD3 antibody is administered for 14 days following administration of the genetically-modified cells. In some embodiments, the anti-CD3 antibody is administered for 15 days following administration of the genetically-modified cells. In some embodiments, the anti-CD3 antibody is administered for 16 days following administration of the genetically-modified cells. In some embodiments, the anti-CD3 antibody is administered for 17 days following administration of the genetically-modified cells. In some embodiments, the anti-CD3 antibody is administered for 18 days following administration of the genetically-modified cells. In some embodiments, the anti-CD3 antibody is administered for 19 days following administration of the genetically-modified cells. In some embodiments, the anti-CD3 antibody is administered for 20 days following administration of the genetically-modified cells. In some embodiments, the anti-CD3 antibody is administered for 21 days following administration of the genetically-modified cells. In some embodiments, the anti-CD3 antibody is administered for 22 days following administration of the genetically-modified cells. In some embodiments, the anti-CD3 antibody is administered for 23 days following administration of the genetically-modified cells. In some embodiments, the anti-CD3 antibody is administered for 24 days following administration of the genetically-modified cells. In some embodiments, the anti-CD3 antibody is administered for 25 days following administration of the genetically-modified cells. In some embodiments, the anti-CD3 antibody is administered for 26 days following administration of the genetically-modified cells. In some embodiments, the anti-CD3 antibody is administered for 27 days following administration of the genetically-modified cells. In some embodiments, the anti-CD3 antibody is administered for 28 days following administration of the genetically-modified cells. In some embodiments, the anti-CD3 antibody is administered for 29 days following administration of the genetically-modified cells. In some embodiments, the anti-CD3 antibody is administered for 30 days following administration of the genetically-modified cells. In some embodiments, the anti-CD3 antibody is administered for 1 month following administration of the genetically-modified cells. In some embodiments, the anti-CD3 antibody is administered for 2 months following administration of the genetically-modified cells. In some embodiments, the anti-CD3 antibody is administered for 3 months following administration of the genetically-modified cells. In some embodiments, the anti-CD3 antibody is administered for 4 months following administration of the genetically-modified cells. In some embodiments, the anti-CD3 antibody is administered for 5 months following administration of the genetically-modified cells. In some embodiments, the anti-CD3 antibody is administered for 6 months following administration of the genetically-modified cells. In some embodiments, the anti-CD3 antibody is administered for 7 months following administration of the genetically-modified cells. In some embodiments, the anti-CD3 antibody is administered for 8 months following administration of the genetically-modified cells. In some embodiments, the anti-CD3 antibody is administered for 9 months following administration of the genetically-modified cells. In some embodiments, the anti-CD3 antibody is administered for 10 months following administration of the genetically-modified cells. In some embodiments, the anti-CD3 antibody is administered for 11 months following administration of the genetically-modified cells. In some embodiments, the anti-CD3 antibody is administered for 12 months following administration of the genetically-modified cells.

In some embodiments, the individual dosage of anti-CD3 antibody may be administered to a subject 1×, 2×, 3×, 4× or more per day. In some embodiments, the anti-CD3 antibody is administered to a subject 1× per day. In some embodiments, the anti-CD3 antibody is administered to a subject 2× per day. In some embodiments, the anti-CD3 antibody is administered to a subject 3× per day. In some embodiments, the anti-CD3 antibody is administered to a subject 4× per day. In some embodiments, the anti-CD3 antibody is administered to a subject continuously.

To achieve lymphodepletion, the anti-CD3 antibody or antigen binding fragment thereof can be administered alone or in combination with an additional lymphodepletion agent described herein (e.g., a lymphodepleting chemotherapeutic agent such as fludarabine and cyclophosphamide). In some embodiments, 1, 2, 3, 4 or more additional lymphodepleting agents may be administered in the lymphodepletion regimen that includes an anti-CD3 antibody or antigen binding fragment thereof.

In some embodiments, the anti-CD3 antibody or antigen binding fragment thereof is administered prior to administration of an additional lymphodepletion agent (e.g., a lymphodepleting chemotherapeutic agent). For example, in some embodiments, the anti-CD3 antibody or antigen binding fragment thereof is administered from 1 hour to 1 month or more prior to administration of an additional lymphodepletion agent (e.g., a lymphodepleting chemotherapeutic agent). For example, the anti-CD3 antibody or antigen binding fragment thereof can be administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, or more, prior to administration of the additional lymphodepletion agent.

In other embodiments, the anti-CD3 antibody or antigen binding fragment thereof is administered following administration of an additional lymphodepletion agent (e.g., a lymphodepleting chemotherapeutic agent). For example, in some embodiments, the anti-CD3 antibody or antigen binding fragment thereof is administered from 1 hour to 1 month or more (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more) following administration of the additional lymphodepleting agent.

In other embodiments, the anti-CD3 antibody or antigen binding fragment thereof is administered in conjunction with the an additional lymphodepletion agent (e.g., a lymphodepleting chemotherapeutic agent). That is, the anti-CD3 antibody or antigen binding fragment thereof is administered concurrently with the additional lymphodepletion agent(s).

To promote lymphodepletion prior to administration of the genetically-modified cells provided herein, an additional lymphodepletion agent (e.g., a lymphodepleting chemotherapeutic agent) may be administered to the subject prior to administration of the genetically-modified cells. For example, the lymphodepletion agent (e.g., a lymphodepleting chemotherapeutic agent) can be administered one day to one month (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days) prior to administration of the genetically-modified cells. In some embodiments, a lymphodepleting chemotherapeutic agent is administered to the subject three or more days prior to administration of the genetically-modified cells. In certain embodiments, administration of a lymphodepleting chemotherapeutic agent ends at least one to two days prior to administration of the genetically-modified cells.

In some embodiments, a lymphodepleting chemotherapeutic agent can be administered as a single dose per day on each of eight consecutive days, as a single dose per day on each of seven consecutive days, as a single dose per day on each of six consecutive days, as a single dose per day on each of five consecutive days, as a single dose per day on each of four consecutive days, as a single dose per day on each of three consecutive days, as a single dose per day on each of two consecutive days, or as a single dose on one day.

In some embodiments, the lymphodepletion agent is administered daily starting 14 days prior to administration of the genetically-modified cells and ending 1 day prior to administration of the genetically-modified cells. In some embodiments, the lymphodepletion agent is administered daily starting 13 days prior to administration of the genetically-modified cells and ending 1 day prior to administration of the genetically-modified cells. In some embodiments, the lymphodepletion agent is administered daily starting 12 days prior to administration of the genetically-modified cells and ending 1 day prior to administration of the genetically-modified cells. In some embodiments, the lymphodepletion agent is administered daily starting 11 days prior to administration of the genetically-modified cells and ending 1 day prior to administration of the genetically-modified cells. In some embodiments, the lymphodepletion agent is administered daily starting 10 days prior to administration of the genetically-modified cells and ending 1 day prior to administration of the genetically-modified cells. In some embodiments, the lymphodepletion agent is administered daily starting 9 days prior to administration of the genetically-modified cells and ending 1 day prior to administration of the genetically-modified cells. In some embodiments, the lymphodepletion agent is administered daily starting 8 days prior to administration of the genetically-modified cells and ending 1 day prior to administration of the genetically-modified cells. In some embodiments, the lymphodepletion agent is administered daily starting 7 days prior to administration of the genetically-modified cells and ending 1 day prior to administration of the genetically-modified cells. In some embodiments, the lymphodepletion agent is administered daily starting 6 days prior to administration of the genetically-modified cells and ending 1 day prior to administration of the genetically-modified cells. In some embodiments, the lymphodepletion agent is administered daily starting 5 days prior to administration of the genetically-modified cells and ending 1 day prior to administration of the genetically-modified cells. In some embodiments, the lymphodepletion agent is administered daily starting 4 days prior to administration of the genetically-modified cells and ending 1 day prior to administration of the genetically-modified cells. In some embodiments, the lymphodepletion agent is administered daily starting 3 days prior to administration of the genetically-modified cells and ending 1 day prior to administration of the genetically-modified cells. In some embodiments, the lymphodepletion agent is administered daily starting 2 days prior to administration of the genetically-modified cells and ending 1 day prior to administration of the genetically-modified cells. In some embodiments, the lymphodepletion agent is administered 1 day prior to administration of the genetically-modified cells.

In some embodiments, the lymphodepletion agent is administered daily starting 14 days prior to administration of the genetically-modified cells and ending 2 days prior to administration of the genetically-modified cells. In some embodiments, the lymphodepletion agent is administered daily starting 13 days prior to administration of the genetically-modified cells and ending 2 days prior to administration of the genetically-modified cells. In some embodiments, the lymphodepletion agent is administered daily starting 12 days prior to administration of the genetically-modified cells and ending 2 days prior to administration of the genetically-modified cells. In some embodiments, the lymphodepletion agent is administered daily starting 11 days prior to administration of the genetically-modified cells and ending 2 days prior to administration of the genetically-modified cells. In some embodiments, the lymphodepletion agent is administered daily starting 10 days prior to administration of the genetically-modified cells and ending 2 days prior to administration of the genetically-modified cells. In some embodiments, the lymphodepletion agent is administered daily starting 9 days prior to administration of the genetically-modified cells and ending 2 days prior to administration of the genetically-modified cells. In some embodiments, the lymphodepletion agent is administered daily starting 8 days prior to administration of the genetically-modified cells and ending 2 days prior to administration of the genetically-modified cells. In some embodiments, the lymphodepletion agent is administered daily starting 7 days prior to administration of the genetically-modified cells and ending 2 days prior to administration of the genetically-modified cells. In some embodiments, the lymphodepletion agent is administered daily starting 6 days prior to administration of the genetically-modified cells and ending 2 days prior to administration of the genetically-modified cells. In some embodiments, the lymphodepletion agent is administered daily starting 5 days prior to administration of the genetically-modified cells and ending 2 days prior to administration of the genetically-modified cells. In some embodiments, the lymphodepletion agent is administered daily starting 4 days prior to administration of the genetically-modified cells and ending 2 days prior to administration of the genetically-modified cells. In some embodiments, the lymphodepletion agent is administered daily starting 3 days prior to administration of the genetically-modified cells and ending 2 days prior to administration of the genetically-modified cells. In some embodiments, the lymphodepletion agent is administered 2 days prior to administration of the genetically-modified cells.

In some embodiments, the lymphodepletion agent is administered daily starting 14 days prior to administration of the genetically-modified cells and ending 3 days prior to administration of the genetically-modified cells. In some embodiments, the lymphodepletion agent is administered daily starting 13 days prior to administration of the genetically-modified cells and ending 3 days prior to administration of the genetically-modified cells. In some embodiments, the lymphodepletion agent is administered daily starting 12 days prior to administration of the genetically-modified cells and ending 3 days prior to administration of the genetically-modified cells. In some embodiments, the lymphodepletion agent is administered daily starting 11 days prior to administration of the genetically-modified cells and ending 3 days prior to administration of the genetically-modified cells. In some embodiments, the lymphodepletion agent is administered daily starting 10 days prior to administration of the genetically-modified cells and ending 3 days prior to administration of the genetically-modified cells. In some embodiments, the lymphodepletion agent is administered daily starting 9 days prior to administration of the genetically-modified cells and ending 3 days prior to administration of the genetically-modified cells. In some embodiments, the lymphodepletion agent is administered daily starting 8 days prior to administration of the genetically-modified cells and ending 3 days prior to administration of the genetically-modified cells. In some embodiments, the lymphodepletion agent is administered daily starting 7 days prior to administration of the genetically-modified cells and ending 3 days prior to administration of the genetically-modified cells. In some embodiments, the lymphodepletion agent is administered daily starting 6 days prior to administration of the genetically-modified cells and ending 3 days prior to administration of the genetically-modified cells. In some embodiments, the lymphodepletion agent is administered daily starting 5 days prior to administration of the genetically-modified cells and ending 3 days prior to administration of the genetically-modified cells. In some embodiments, the lymphodepletion agent is administered daily starting 4 days prior to administration of the genetically-modified cells and ending 3 days prior to administration of the genetically-modified cells. In some embodiments, the lymphodepletion agent is administered 3 days prior to administration of the genetically-modified cells.

In some embodiments, the lymphodepletion agent is administered daily starting 14 days prior to administration of the genetically-modified cells and ending 4 days prior to administration of the genetically-modified cells. In some embodiments, the lymphodepletion agent is administered daily starting 13 days prior to administration of the genetically-modified cells and ending 4 days prior to administration of the genetically-modified cells. In some embodiments, the lymphodepletion agent is administered daily starting 12 days prior to administration of the genetically-modified cells and ending 4 days prior to administration of the genetically-modified cells. In some embodiments, the lymphodepletion agent is administered daily starting 11 days prior to administration of the genetically-modified cells and ending 4 days prior to administration of the genetically-modified cells. In some embodiments, the lymphodepletion agent is administered daily starting 10 days prior to administration of the genetically-modified cells and ending 4 days prior to administration of the genetically-modified cells. In some embodiments, the lymphodepletion agent is administered daily starting 9 days prior to administration of the genetically-modified cells and ending 4 days prior to administration of the genetically-modified cells. In some embodiments, the lymphodepletion agent is administered daily starting 8 days prior to administration of the genetically-modified cells and ending 4 days prior to administration of the genetically-modified cells. In some embodiments, the lymphodepletion agent is administered daily starting 7 days prior to administration of the genetically-modified cells and ending 4 days prior to administration of the genetically-modified cells. In some embodiments, the lymphodepletion agent is administered daily starting 6 days prior to administration of the genetically-modified cells and ending 4 days prior to administration of the genetically-modified cells. In some embodiments, the lymphodepletion agent is administered daily starting 5 days prior to administration of the genetically-modified cells and ending 4 days prior to administration of the genetically-modified cells. In some embodiments, the lymphodepletion agent is administered 4 days prior to administration of the genetically-modified cells.

In some embodiments, the lymphodepletion agent is administered every other day starting 13 days prior to administration of the genetically-modified cells and ending 1 day prior to administration of the genetically-modified cells. In some embodiments, the lymphodepletion agent is administered every other day starting 11 days prior to administration of the genetically-modified cells and ending 1 day prior to administration of the genetically-modified cells. In some embodiments, the lymphodepletion agent is administered every other day starting 9 days prior to administration of the genetically-modified cells and ending 1 day prior to administration of the genetically-modified cells. In some embodiments, the lymphodepletion agent is administered every other day starting 7 days prior to administration of the genetically-modified cells and ending 1 day prior to administration of the genetically-modified cells. In some embodiments, the lymphodepletion agent is administered every other day starting 5 days prior to administration of the genetically-modified cells and ending 1 day prior to administration of the genetically-modified cells. In some embodiments, the lymphodepletion agent is administered every other day starting 3 days prior to administration of the genetically-modified cells and ending 1 day prior to administration of the genetically-modified cells.

In some embodiments, the lymphodepletion agent is administered every other day starting 13 days prior to administration of the genetically-modified cells and ending 3 days prior to administration of the genetically-modified cells. In some embodiments, the lymphodepletion agent is administered every other day starting 11 days prior to administration of the genetically-modified cells and ending 3 days prior to administration of the genetically-modified cells. In some embodiments, the lymphodepletion agent is administered every other day starting 9 days prior to administration of the genetically-modified cells and ending 3 days prior to administration of the genetically-modified cells. In some embodiments, the lymphodepletion agent is administered every other day starting 7 days prior to administration of the genetically-modified cells and ending 3 days prior to administration of the genetically-modified cells. In some embodiments, the lymphodepletion agent is administered every other day starting 5 days prior to administration of the genetically-modified cells and ending 3 days prior to administration of the genetically-modified cells.

In some embodiments, the lymphodepletion agent is administered every other day starting 14 days prior to administration of the genetically-modified cells and ending 2 days prior to administration of the genetically-modified cells. In some embodiments, the lymphodepletion agent is administered every other day starting 12 days prior to administration of the genetically-modified cells and ending 2 days prior to administration of the genetically-modified cells. In some embodiments, the lymphodepletion agent is administered every other day starting 10 days prior to administration of the genetically-modified cells and ending 2 days prior to administration of the genetically-modified cells. In some embodiments, the lymphodepletion agent is administered every other day starting 8 days prior to administration of the genetically-modified cells and ending 2 days prior to administration of the genetically-modified cells. In some embodiments, the lymphodepletion agent is administered every other day starting 6 days prior to administration of the genetically-modified cells and ending 2 days prior to administration of the genetically-modified cells. In some embodiments, the lymphodepletion agent is administered every other day starting 4 days prior to administration of the genetically-modified cells and ending 2 days prior to administration of the genetically-modified cells.

In some embodiments, the lymphodepletion agent is administered every other day starting 14 days prior to administration of the genetically-modified cells and ending 4 days prior to administration of the genetically-modified cells. In some embodiments, the lymphodepletion agent is administered every other day starting 12 days prior to administration of the genetically-modified cells and ending 4 days prior to administration of the genetically-modified cells. In some embodiments, the lymphodepletion agent is administered every other day starting 10 days prior to administration of the genetically-modified cells and ending 4 days prior to administration of the genetically-modified cells. In some embodiments, the lymphodepletion agent is administered every other day starting 8 days prior to administration of the genetically-modified cells and ending 4 days prior to administration of the genetically-modified cells. In some embodiments, the lymphodepletion agent is administered every other day starting 6 days prior to administration of the genetically-modified cells and ending 4 days prior to administration of the genetically-modified cells.

In some embodiments, the lymphodepleting chemotherapeutic agent is cyclophosphamide, which is administered as a single dose per day on each of five consecutive days, as a single dose per day on each of four consecutive days, as a single dose per day on each of three consecutive days, as a single dose per day on each of two consecutive days, or as a single dose on one day. In certain embodiments, the cyclophosphamide is administered as one dose per day for three consecutive days or one dose per day for two consecutive days. In certain embodiments, administration of cyclophosphamide ends at least one to three days prior to administration of the genetically-modified cells.

In some embodiments, the lymphodepleting chemotherapeutic agent is fludarabine, which is administered as a single dose per day on each of five consecutive days, as a single dose per day on each of four consecutive days, as a single dose per day on each of three consecutive days, as a single dose per day on each of two consecutive days, or as a single dose on one day. In other embodiments, the fludarabine is administered as one dose per day for five consecutive days or as one dose per day for three consecutive days. In certain embodiments, administration of fludarabine ends at least one to two days prior to administration of the genetically-modified cells.

In some embodiments, cyclophosphamide is administered to the subject daily starting five days and ending three days prior to administration of the composition comprising the genetically-modified cells. In some embodiments, cyclophosphamide is administered to the subject daily starting five days and ending two days prior to administration of the composition comprising the genetically-modified cells. In some embodiments, cyclophosphamide is administered to the subject daily starting four days and ending three days prior to administration of the composition comprising the genetically-modified cells. In some embodiments, cyclophosphamide is administered to the subject daily starting four days and ending two days prior to administration of the composition comprising the genetically-modified cells.

In particular embodiments, fludarabine is administered to the subject daily starting five days and ending three days prior to administration of the composition comprising the genetically-modified cells. In some embodiments, fludarabine is administered to the subject daily starting five days and ending two days prior to administration of the composition comprising the genetically-modified cells. In other particular embodiments, fludarabine is administered to the subject daily starting seven days and ending three days prior to administration of the composition comprising the genetically-modified cells. In other particular embodiments, fludarabine is administered to the subject daily starting seven days and ending two days prior to administration of the composition comprising the genetically-modified cells.

In certain embodiments, cyclophosphamide is administered to the subject daily starting five days and ending three days prior to administration of the composition comprising the genetically-modified cells, and fludarabine is administered to the subject daily starting five days and ending three days prior to administration of the composition comprising the genetically-modified cells. In some embodiments, cyclophosphamide is administered to the subject daily starting five days and ending two days prior to administration of the composition comprising the genetically-modified cells, and fludarabine is administered to the subject daily starting five days and ending two days prior to administration of the composition comprising the genetically-modified cells.

In other embodiments, cyclophosphamide is administered to the subject daily starting four days and ending three days prior to administration of the composition comprising the genetically-modified cells, and fludarabine is administered to the subject at a dose daily starting seven days and ending three days prior to administration of the composition comprising the genetically-modified cells. In other embodiments, cyclophosphamide is administered to the subject daily starting four days and ending two days prior to administration of the composition comprising the genetically-modified cells, and fludarabine is administered to the subject at a dose daily starting seven days and ending two days prior to administration of the composition comprising the genetically-modified cells.

In some embodiments, the additional lymphodepleting chemotherapeutic agent is melphalan. Suitable dosing for melphalan is known in the art. Melphalan may be administered in an amount of about 1 mg/day to about 30 mg/day per single dose (see prescribing information for Alkeran® (NDA)). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is bendamustine. Suitable dosing for bendamustine is known in the art. Bendamustine may be administered in an amount of about 10 mg/m²/day to about 200 mg/m²/day (see prescribing information for bendamustine). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is mercaptopurine. Suitable dosing for mercaptopurine is known in the art. Mercaptopurine may be administered in an amount of about 0.5 to about 5 mg/kg/day (see prescribing information for Purinethol®). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is daunorubicin. Suitable dosing for daunorubicin is known in the art. Daunorubicin may be administered in an amount of about 10 to about 500 mg/m²/day (see prescribing information for daunorubicin). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is cytarabine. Suitable dosing for cytarabine is known in the art (see prescribing information for DepoCyt®). Cytarabine may be administered in an amount of about 1 mg/day to about 100 mg/day. Each individual dosage may be given 1, 2, 3, 4 or more times a day consecutively for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is L-asparaginase. Suitable dosing for L-asparaginase is known in the art (see prescribing information for Elspar®). L-asparaginase may be administered in an amount of about 100 I.U./kg/day to about 1,500 I.U./kg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is methotrexate. Suitable dosing for methotrexate is known in the art (see prescribing information for methotrexate). Methotrexate may be administered in an amount of about 1 mg/m²/day to about 10 mg/m²/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is prednisolone or prednisone. Suitable dosing for methotrexate is known in the art (see prescribing information for Prapred ODT®). Prednisolone or prednisone may be administered in an amount of about 1 mg/day to about 100 mg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is prednisolone or prednisone. Suitable dosing for prednisolone or prednisone is known in the art (see prescribing information for Oprapred ODT®). Prednisolone or prednisone may be administered in an amount of about 1 mg/day to about 100 mg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting chemotherapeutic agent is nelarabine. Suitable dosing for nelarabine is known in the art (see prescribing information for ARRANON®). Nelarabine may be administered in an amount of about 500 mg/m²/day to about 2000 mg/m²/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting agent is rituximab. Suitable dosing for rituximab is known in the art (see prescribing information for Rituxan®). Rituximab may be administered in an amount of about 100 mg/m²/day to about 3000 mg/m²/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In some embodiments, the additional lymphodepleting agent is alemtuzumab. Suitable dosing for alemtuzumab is known in the art (see prescribing information for Campath®). Alemtuzumab may be administered in an amount of about 1 mg/day to about 30 mg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

In the present invention, the dose of lymphodepleting chemotherapeutic agent can be adjusted depending on the desired effect, e.g., to modulate the reduction of endogenous lymphocytes and/or control the severity of adverse events.

For example, in embodiments where the lymphodepleting chemotherapeutic agent is cyclophosphamide, the dose of cyclophosphamide can be higher than about 250 mg/m²/day and lower than about 1500 mg/m²/day. In some embodiments, the dose of cyclophosphamide is about 250-1500 mg/m²/day, about 300-1500 mg/m²/day, about 350-1500 mg/m²/day, about 400-1500 mg/m²/day, about 450-1500 mg/m²/day, about 500-1500 mg/m²/day, about 550-1500 mg/m²/day, or about 600-1500 mg/m²/day. In another embodiment, the dose of cyclophosphamide is about 250-1500 mg/m²/day, about 350-1000 mg/m²/day, about 400-900 mg/m²/day, about 450-800 mg/m²/day, about 450-700 mg/m²/day, about 450-600 mg/m²/day, or about 450-550 mg/m²/day. In certain embodiments, the dose of cyclophosphamide is about 250 mg/m²/day, about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day. In one particular embodiment, the dose of cyclophosphamide is about 500 mg/m²/day. In one particular embodiment, the dose of cyclophosphamide is about 1000 mg/m²/day.

In the present invention, the dose of fludarabine can also be adjusted depending on the desired effect. For example, the dose of fludarabine can be higher than 10 mg/m²/day and lower than 100 mg/m²/day. In some embodiments, the dose of fludarabine is about 10-100 mg/m²/day, about 15-100 mg/m²/day, about 20-100 mg/m²/day, about 25-900 mg/m²/day, about 30-900 mg/m²/day, about 35-100 mg/m²/day, about 40-100 mg/m²/day, about 45-100 mg/m²/day, about 50-100 mg/m²/day, about 55-100 mg/m²/day, or about 60-100 mg/m²/day. In other embodiments, the dose of fludarabine is about 10-100 mg/m²/day, about 10-90 mg/m²/day, about 10-80 mg/m²/day, about 10-70 mg/m²/day, about 10-60 mg/m²/day, about 10-50 mg/m²/day, about 10-45 mg/m²/day, about 20-40 mg/m²/day, about 25-35 mg/m²/day, or about 28-32 mg/m²/day. In certain embodiments, the dose of fludarabine is about 10 mg/m²/day, 15 mg/m²/day, 20 mg/m²/day, 25 mg/m²/day, 30 mg/m²/day, 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day. In one particular embodiment, the dose of fludarabine is about 30 mg/m²/day.

In some embodiments, the dose of cyclophosphamide is about 250-1500 mg/m²/day and the dose of fludarabine is about 10-100 mg/m²/day. In certain embodiments, the dose of cyclophosphamide is about 500 mg/m²/day and the dose of fludarabine is about 30 mg/m²/day. In other particular embodiments, the dose of cyclophosphamide is about 1000 mg/m²/day and the dose of fludarabine is about 30 mg/m²/day.

In particular embodiments, cyclophosphamide is administered to the subject at a dose of about 500 mg/m²/day daily starting five days and ending three days prior to administration of the composition comprising the genetically-modified cells. In particular embodiments, cyclophosphamide is administered to the subject at a dose of about 500 mg/m²/day daily starting five days and ending two days prior to administration of the composition comprising the genetically-modified cells. In other particular embodiments, cyclophosphamide is administered to the subject at a dose of about 1000 mg/m²/day daily starting four days and ending three days prior to administration of the composition comprising the genetically-modified cells. In other particular embodiments, cyclophosphamide is administered to the subject at a dose of about 1000 mg/m²/day daily starting four days and ending two days prior to administration of the composition comprising the genetically-modified cells.

In particular embodiments, fludarabine is administered to the subject at a dose of about 30 mg/m²/day daily starting five days and ending three days prior to administration of the composition comprising the genetically-modified cells. In particular embodiments, fludarabine is administered to the subject at a dose of about 30 mg/m²/day daily starting five days and ending two days prior to administration of the composition comprising the genetically-modified cells. In other particular embodiments, fludarabine is administered to the subject at a dose of about 30 mg/m²/day daily starting seven days and ending three days prior to administration of the composition comprising the genetically-modified cells. In other particular embodiments, fludarabine is administered to the subject at a dose of about 30 mg/m²/day daily starting seven days and ending two days prior to administration of the composition comprising the genetically-modified cells. In certain embodiments, cyclophosphamide is administered to the subject at a dose of about 500 mg/m²/day daily starting five days and ending three days prior to administration of the composition comprising the genetically-modified cells, and fludarabine is administered to the subject at a dose of about 30 mg/m²/day daily starting five days and ending three days prior to administration of the composition comprising the genetically-modified cells. In certain embodiments, cyclophosphamide is administered to the subject at a dose of about 500 mg/m²/day daily starting five days and ending two days prior to administration of the composition comprising the genetically-modified cells, and fludarabine is administered to the subject at a dose of about 30 mg/m²/day daily starting five days and ending two days prior to administration of the composition comprising the genetically-modified cells.

In yet further embodiments, cyclophosphamide is administered to the subject at a dose of about 1000 mg/m²/day daily starting four days and ending three days prior to administration of the composition comprising the genetically-modified cells, and fludarabine is administered to the subject at a dose of about 30 mg/m²/day daily starting seven days and ending three days prior to administration of the composition comprising the genetically-modified cells. In yet further embodiments, cyclophosphamide is administered to the subject at a dose of about 1000 mg/m²/day daily starting four days and ending two days prior to administration of the composition comprising the genetically-modified cells, and fludarabine is administered to the subject at a dose of about 30 mg/m²/day daily starting seven days and ending two days prior to administration of the composition comprising the genetically-modified cells.

It is understood that reference to dosing of a CD3-specific antibody encompasses dosing of an antigen binding fragment thereof.

### 2.5 Pharmaceutical Compositions

In another aspect of the invention, the present disclosure provides a pharmaceutical composition comprising a lymphodepleting chemotherapeutic agent, an antibody or antigen binding fragment thereof that specifically binds CD3 (i.e., an anti-CD3 antibody or antigen binding fragment thereof), and a pharmaceutically-acceptable carrier. The invention also provides pharmaceutical compositions comprising a pharmaceutically-acceptable carrier and a genetically-modified immune cell, or population of genetically-modified immune cells, described herein.

Such pharmaceutical compositions can be prepared in accordance with known techniques. See, e.g., Remington, The Science and Practice of Pharmacy (21^{st} ed. 2005). In the manufacture of a pharmaceutical formulation, according to the present disclosure, cells are typically admixed with a pharmaceutically acceptable carrier and the resulting composition is administered to a subject (e.g., a human). The pharmaceutically acceptable carrier must, of course, be acceptable in the sense of being compatible with any other ingredients in the formulation and must not be deleterious to the subject. In some embodiments, the pharmaceutical compositions of the present disclosure further comprise one or more additional agents useful in the treatment of a disease (e.g., cancer) in a subject.

The present disclosure also provides genetically-modified cells (e.g., T cells modified to express a chimeric antigen receptor (CAR) or an exogenous T cell receptor (TCR) and do not express CD3 on the cell surface), or populations thereof, described herein for use as a medicament. The present disclosure further provides the use of genetically-modified cells or populations thereof described herein in the manufacture of a medicament for treating a disease in a subject in need thereof. In one such aspect, the medicament is useful for cancer immunotherapy in subjects in need thereof.

In some embodiments, the pharmaceutical compositions and medicaments of the present disclosure are useful for treating any disease state that can be targeted by T cell adoptive immunotherapy. In a particular embodiment, the pharmaceutical compositions and medicaments of the present disclosure are useful as immunotherapy in the treatment of cancer. Non-limiting examples of cancers which may be treated with the pharmaceutical compositions and medicaments of the present disclosure are carcinomas, lymphomas, sarcomas, melanomas, blastomas, leukemias, myelomas, and germ cell tumors, including but not limited to cancers of B-cell origin, neuroblastoma, osteosarcoma, prostate cancer, renal cell carcinoma, rhabdomyosarcoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, and B-cell non-Hodgkin lymphoma. In some examples, cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

### 2.6 Kits

In another aspect of the invention, a kit containing materials useful for the treatment regimens, e.g., for lymphodepletion and/or the treatment of a cancer, is provided. In some embodiments, the kit includes an anti-CD3 antibody, or antigen-binding fragment thereof, packaged in combination with a composition comprising a population of genetically-modified cells described herein (e.g., T cells modified to express a chimeric antigen receptor (CAR) or an exogenous T cell receptor (TCR) and do not express CD3 on the cell surface) and/or a lymphodepletion agent (e.g., a lymphodepleting chemotherapeutic agent such as fludarabine and/or cyclophosphamide) as a kit. The kit can further include optional components that aid in the administration of the unit dose to subjects, such as vials for reconstituting powder forms, syringes for injection, customized IV delivery systems, etc. Additionally, the unit dose kit can contain instructions for preparation and administration of the compositions.

In certain embodiments, the kit includes an antibody, or antigen-binding fragment thereof, that specifically binds CD3, and a composition comprising a population of genetically-modified T cells described herein (e.g., T cells modified to express a chimeric antigen receptor (CAR) or an exogenous T cell receptor (TCR) and do not express CD3 on the cell surface). In other embodiments, the kit includes a lymphodepleting chemotherapeutic agent, an antibody, or antigen-binding fragment thereof, that specifically binds CD3, and a composition comprising a population of genetically-modified T cells described herein.

In addition, the kit may comprise a package inserts with instructions for use. For example, the instructions for use may instruct the user of the composition to administer the anti-CD3 antibody composition to the subject with a lymphodepleting chemotherapeutic agent (e.g., fludarabine, cyclophosphamide, or a combination thereof) and/or a composition including genetically-modified cell described herein (e.g., T cells modified to express a chimeric antigen receptor (CAR) or an exogenous T cell receptor (TCR) and do not express CD3 on the cell surface). In certain embodiments, the instructions for use instruct the user of the composition to administer the anti-CD3 antibody composition in combination with a chemotherapeutic agent to a subject having a cancer in an amount effective to achieve lymphodepletion prior to or concurrently with administration of the genetically-modified cells described herein.

The kit may be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

### 2.7 Chimeric Antigen Receptors and Exogenous T Cell Receptors

Provided herein are genetically-modified cells expressing a chimeric antigen receptor (CAR) or an exogenous T cell receptor (TCR). Generally, a CAR of the present disclosure will comprise at least an extracellular domain, a transmembrane domain, and an intracellular domain. In some embodiments, the extracellular domain comprises a target-specific binding element otherwise referred to as a ligand-binding domain or moiety. In some embodiments, the intracellular domain, or cytoplasmic domain, comprises at least one co-stimulatory domain and one or more signaling domains such as, for example, CD3ζ.

In some embodiments, a CAR useful in the invention comprises an extracellular, target-specific binding element otherwise referred to as a ligand-binding domain or moiety. The choice of ligand-binding domain depends upon the type and number of ligands that define the surface of a target cell. For example, the ligand-binding domain may be chosen to recognize a ligand that acts as a cell surface marker on target cells associated with a particular disease state. Thus, examples of cell surface markers that may act as ligands for the ligand-binding domain in a CAR can include those associated with viruses, bacterial and parasitic infections, autoimmune disease, and cancer cells. In some embodiments, a CAR is engineered to target a tumor-specific antigen of interest by way of engineering a desired ligand-binding moiety that specifically binds to an antigen on a tumor cell. In the context of the present disclosure, “tumor antigen” or “tumor-specific antigen” refer to antigens that are common to specific hyperproliferative disorders such as cancer.

In some embodiments, the extracellular ligand-binding domain of the CAR is specific for any antigen or epitope of interest, particularly any cancer antigen or epitope of interest. As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79B, ILIRAP, glypican 3 (GPC3), CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, insulin growth factor (IGFl)-1, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the Al domain of tenascin-C (TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), CS1, or a virus-specific surface antigen such as an HIV-specific antigen (such as HIV gpl20); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

In some embodiments, the extracellular domain of a chimeric antigen receptor further comprises an autoantigen (see, Payne et al. (2016) Science, Vol. 353 (6295): 179-184), which can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs).

In some embodiments, the extracellular domain of a chimeric antigen receptor can comprise a naturally-occurring ligand for an antigen of interest, or a fragment of a naturally-occurring ligand which retains the ability to bind the antigen of interest.

In some embodiments, a CAR comprises a transmembrane domain which links the extracellular ligand-binding domain or autoantigen with the intracellular signaling and co-stimulatory domains via a hinge or spacer sequence. The transmembrane domain can be derived from any membrane-bound or transmembrane protein. For example, the transmembrane polypeptide can be a subunit of the T-cell receptor (i.e., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. Alternatively, the transmembrane domain can be synthetic and can comprise predominantly hydrophobic residues such as leucine and valine. In particular examples, the transmembrane domain is a CD8α transmembrane polypeptide (e.g., SEQ ID NO: 10).

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain (e.g., SEQ ID NO: 11).

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. An intracellular signaling domain, such as CD3ζ (SEQ ID NO: 8) can provide an activation signal to the cell in response to binding of the extracellular domain. As discussed, the activation signal can induce an effector function of the cell such as, for example, cytolytic activity or cytokine secretion.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such TRAF binding-domains may include, for example, those set forth in SEQ ID NOs: 3-5. Such intracellular co-stimulatory domains can be any of those known in the art and can include, without limitation, those co-stimulatory domains disclosed in WO 2018/067697 including, for example, Novel 6 (“N6”; SEQ ID NO: 6). Further examples of co-stimulatory domains can include 4-1BB (CD137; SEQ ID NO: 7), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

The intracellular domains of a chimeric antigen receptor as disclosed herein may be linked to each other in a specified or random order. In certain embodiments, the intracellular domain of a chimeric antigen receptor as disclosed herein may contain short polypeptide linker or spacer regions, between 2 to 30 amino acids in length. In other embodiments, the intracellular domain of a chimeric antigen receptor as disclosed herein may contain short polypeptide linker or spacer regions, between 2 to 10 amino acids in length. In some embodiments, the linker or spacer regions may include an amino acid sequence that substantially comprises glycine and serine.

The CAR can be specific for any type of cancer cell. Such cancers can include, without limitation, any of those cancers described elsewhere herein.

It is to be understood that a CAR as disclosed herein can include a domain (e.g., an extracellular domain, a transmembrane domain, an intracellular (cytoplasmic) domain, a signaling domain, or any combination thereof) having a sequence as set forth herein, or a variant thereof, or a fragment thereof, of any one or more of the domains disclosed herein (e.g., a variant and/or fragment that retains the function required for the chimeric antigen receptor activity). In some embodiments, a variant has 1, 2, 3, 4, 5, 6, 7, 8, 9, or 10 amino acid changes relative to the sequence. In some embodiments, a variant has a sequence that is at least 80%, at least 85%, at least 90%, 90%-95%, at least 95% or at least 99% identical to the native or wild-type sequence, or of the sequence provided herein. In some embodiments, a fragment is 1-5, 5-10, 10-20, 20-30, 30-40, or 40-50 amino acids shorter than a sequence provided herein. In some embodiments, a fragment is shorter at the N-terminal, C-terminal, or both terminal regions of the sequence provided. In some embodiments, a fragment contains 80%-85%, 85%-90%, 90%-95%, or 95%-99% of the number of amino acids in a sequence provided herein.

Further, it is to be understood that any of the nucleic acids or polynucleotides that are described herein, that encode a chimeric antigen receptor, or variant thereof, as disclosed herein, can be prepared by a routine method, such as recombinant technology. Methods for preparing a chimeric antigen receptor as described herein may involve, in some embodiments, the generation of a nucleic acid that encodes a polypeptide comprising each of the domains of the chimeric antigen receptor, or variant thereof, as disclosed herein, comprising at least an extracellular domain, a transmembrane domain, and an intracellular domain. In a particular embodiment, the nucleic acid encodes an intracellular domain comprising a signaling domain. In another particular embodiment, the nucleic acid encodes an intracellular domain comprising a co-stimulatory signaling domain. In some embodiments, the nucleic acid encodes a hinge region between the extracellular domain and the transmembrane domain.

In other embodiments, the genetically modified cell comprises a nucleic acid sequence encoding an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest.

In some embodiments, genetically-modified cells described herein comprise an inactivated TCR alpha gene, TRAC gene, TCR beta gene, or TRBC gene. Inactivation of these genes to generate genetically-modified cells of the present invention occurs in at least one or both alleles where the gene is being expressed. Accordingly, inactivation of one or both genes prevents expression of the endogenous TCR alpha chain or the endogenous TCR beta chain protein. Expression of these proteins is required for assembly of the endogenous alpha/beta TCR on the cell surface. Thus, inactivation of the TCR alpha gene and/or the TCR beta gene results in genetically-modified cells that have no detectable cell surface expression of the endogenous alpha/beta TCR. The endogenous alpha/beta TCR incorporates CD3. Therefore, cells with an inactivated gene encoding a component of the alpha/beta TCR will, consequently, have no detectable cell surface expression of CD3.

In some examples, the TCR alpha gene, TRAC gene, TCR beta gene, or TRBC gene is inactivated by insertion of a transgene (e.g., a transgene encoding a CAR or an exogenous TCR). Insertion of the transgene disrupts expression of the endogenous TCR alpha chain or TCR beta chain and, therefore, prevents assembly of an endogenous alpha/beta TCR on the T cell surface, and likewise interferes with expression of CD3 on the cell surface. In some examples, a transgene is inserted into the TRAC gene. In a particular example, a transgene is inserted into the TRAC gene at an engineered meganuclease recognition sequence comprising SEQ ID NO: 1. In particular examples, the transgene is inserted into SEQ ID NO: 1 between nucleotide positions 13 and 14.

As used herein, “detectable cell surface expression of an endogenous alpha/beta TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in the examples herein, and, for example, those described in MacLeod et al. (2017).

Similarly, “detectable cell surface expression of CD3” refers to lack of detection of CD3 on the surface of a genetically-modified cell described herein, or population of genetically-modified cells described herein, as detected using standard experimental methods in the art. Methods for detecting cell surface expression of CD3 on an immune cell include those described in MacLeod et al. (2017).

Human immune cells modified by the present invention may require activation prior to introduction of a nuclease and/or an exogenous sequence of interest. For example, immune cells (e.g., T cells) can be contacted with anti-CD3 and anti-CD28 antibodies that are soluble or conjugated to a support (e.g., beads) for a period of time sufficient to activate the cells.

Genetically-modified cells described herein can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

In some of those embodiments wherein the genetically-modified immune cell expresses a CAR or exogenous TCR, such cells have no detectable cell-surface expression of an endogenous T cell receptor (e.g., an alpha/beta T cell receptor). Thus, the invention further provides a population of genetically-modified cells that express a CAR or exogenous TCR and have no detectable cell-surface expression of CD3. Such cells can comprise an inactivated gene encoding a component of the TCR alpha/beta receptor, such as the TCR alpha gene, the TRAC gene, the TCR beta gene, or the TRBC gene. For example, the population can include a plurality of genetically-modified cells of the invention which express a CAR (i.e., are CAR+), or an exogenous T cell receptor (i.e., exoTCR+), and have no detectable cell-surface expression of CD3. In various embodiments of the invention, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified cell as described herein. In a particular example, the population can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, cells that are both CD3− and CAR+. In another particular example, the population can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, cells that are both CD3− and exoTCR+.

### 2.8 Nucleic Acid Molecules

The genetically-modified cells of the invention comprise an exogenous polynucleotide encoding a chimeric antigen receptor (CAR) or an exogenous T cell receptor (TCR). Accordingly, provided herein are nucleic molecules to generate the genetically-modified cells of the invention.

The nucleic acid molecules can include various promoters which drive expression of the chimeric antigen receptor or exogenous TCR. One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Synthetic promoters are also contemplated as part of the present disclosure. For example, in particular embodiments, the promoter driving expression of the chimeric antigen receptor or exogenous TCR is a JeT promoter (see, WO/2002/012514).

In some embodiments, the promoters are selected based on the desired outcome. It is recognized that different applications can be enhanced by the use of different promoters in the expression cassettes to modulate the timing, location and/or level of expression of the polynucleotides disclosed herein. Such expression constructs may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

In order to assess the expression of a CAR or an exogenous T cell receptor in a genetically-modified cell, the nucleic acid molecule of the invention can optionally comprise an epitope which can be used to detect the presence of the encoded cell-surface protein. In some examples described herein, a CAR coding sequence may include a QBend10 epitope and/or EGFR epitope, which allows for detection using an anti-CD34 antibody and/or an anti-EGFR antibody (see, WO2011/056894, WO2013/153391, and WO/2019/070856 each of which is incorporated by reference herein in its entirety).

In other examples, the nucleic acid molecule can also contain either a selectable marker gene or a reporter gene, or both, to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes and fluorescent marker genes.

Also provided herein are vectors comprising the nucleic acid molecules of the present disclosure. In some embodiments, the nucleic acid molecule is cloned into a vector including, but not limited to a plasmid, a phagemid, a phage derivative, an animal virus, or a cosmid. Vectors of particular interest include expression vectors, replication vectors, probe generation vectors, and sequencing vectors.

In other embodiments, nucleic acid molecules of the invention are provided on viral vectors, such as retroviral vectors, lentiviral vectors, adenoviral vectors, and adeno-associated viral (AAV) vectors. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses (AAVs), herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

### 2.9 Genetically-Modified Cells and Populations Thereof

Provided herein are cells that are genetically-modified to contain at least one exogenous polynucleotide sequence. In specific embodiments, the genetically-modified cell comprises an exogenous polynucleotide encoding a chimeric antigen receptor (CAR) or an exogenous T cell receptor (TCR). Further, the genetically-modified cells of the invention are modified to have no detectable CD3 or endogenous TCR on the cell surface, e.g., via insertion of the exogenous polynucleotide in a target gene encoding one or more components of the T cell receptor complex (e.g., a T cell receptor alpha constant region (TRAC) gene or a T cell receptor beta constant (TRBC) region gene).

In certain embodiments of the present disclosure, a nucleic acid molecule or expression cassette which encodes a CAR or an exogenous TCR is present (i.e., integrated) within the genome of the genetically-modified cell. In particular embodiments, an exogenous nucleic acid molecule is inserted into the chromosome of a cell by targeted insertion at a cleavage site produced by a double-strand break, such as that produced by an engineered nuclease.

In one embodiment, genetically-modified cells contain an exogenous nucleic acid or polynucleotide molecule encoding a CAR or an exogenous TCR positioned within the genome of a cell (e.g., a T cell or an NK cell). In some embodiments, the exogenous polynucleotide is positioned within a target gene of the cell. In various examples, the target gene can encode a component of the endogenous alpha/beta TCR, such as the TCR alpha gene, TRAC gene, TCR beta gene, or TRBC gene. In certain embodiments, the exogenous polynucleotide is positioned within an endogenous T cell receptor alpha constant region gene, such as within exon 1 of the TRAC gene (see, for example, PCT/US2016/055472 or PCT/US2016/055492). In other embodiments, the exogenous polynucleotide is positioned within an endogenous TRBC gene. In particular embodiments, insertion of the exogenous polynucleotide in the target gene prevents expression of the full-length polypeptide encoded by the target gene. For example, insertion of the exogenous polynucleotide in a TRAC or a TRBC gene can prevent full-length expression of a TRC alpha or a TRC beta polypeptide, respectively, and thereby prevent assembly of an endogenous T cell receptor complex, including all of or substantially all of CD3 (e.g., greater than 80%, 90%, 95%, or more), on the cell surface of the genetically-modified cells.

In some embodiments, the genetically-modified cells are eukaryotic cells. In certain other embodiments, the cells are human cells. In other embodiments, the genetically-modified cells are immune cells (e.g., T cells, NK cells, macrophages, monocytes, neutrophils, eosinophils, cytotoxic T lymphocytes, regulatory T cells, or any combination thereof). A population of immune cells can be obtained from any source, such as peripheral blood mononuclear cells (PBMCs), bone marrow, tissues such as spleen, lymph node, thymus, or tumor tissue. A source suitable for obtaining the type of cell desired would be evident to one of skill in the art. In some embodiments, the population of immune cells is derived from PBMCs. In some embodiments, the genetically-modified cells are immune cells derived from induced pluripotent stem cells (iPSCs).

In a particular embodiment, the genetically-modified cells are T cells or NK cells, particularly human T cells or human NK cells that are modified to lack detectable cell surface expression of CD3 or an endogenous TCR complex. In some embodiments, the cells are primary T cells or primary NK cells. T cells and NK cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell and NK cell lines available in the art may be used. In some embodiments of the present disclosure, T cells and NK cells are obtained from a unit of blood collected from a subject using any number of techniques known to the skilled artisan. In one embodiment, cells from the circulating blood of an individual are obtained by apheresis. In some embodiments, the T cells or NK cells are derived from iPSCs.

Methods of preparing cells capable of expressing the CARs or the exogenous TCRs, as described herein, and lacking detectable cell surface expression of CD3 or an endogenous TCR complex may comprise expanding the isolated cells ex vivo. Expanding cells may involve any method that results in an increase in the number of cells capable of expressing a CAR or an exogenous TCR, for example, by allowing the cells to proliferate or stimulating the cells to proliferate. Methods for stimulating expansion of cells will depend on the type of cell used for expression of the CAR or the exogenous TCR and will be evident to one of skill in the art. In some embodiments, the cells expressing the CAR or the exogenous TCR, as described herein, and lacking detectable cell surface expression of CD3 or an endogenous TCR complex are expanded ex vivo prior to administration to a subject.

The present disclosure further provides a population of genetically-modified cells comprising a plurality of genetically-modified cells as described herein, which comprise an exogenous polynucleotide encoding a CAR or an exogenous TCR and lack detectable cell surface expression of CD3 or an endogenous TCR complex. Thus, in various embodiments of the invention, a population of genetically-modified cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are genetically-modified cells that comprise a CAR or an exogenous TCR, as disclosed herein, and have no detectable cell surface expression of CD3 or an endogenous TCR complex. In certain embodiments, a population of genetically-modified cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population express a CAR or exogenous TCR, as described herein, and have no detectable cell surface expression of CD3 or an endogenous TCR complex.

### 2.10 Methods for Producing Genetically-Modified Cells

The present disclosure provides methods for producing genetically-modified cells comprising a chimeric antigen receptor (CAR) or an exogenous T cell receptor (TCR) and lacking detectable cell surface expression of CD3 or an endogenous TCR complex. In specific embodiments, methods are provided for modifying a cell to comprise an exogenous polynucleotide encoding a CAR or an exogenous TCR and to have no detectable cell surface expression of CD3 or an endogenous TCR complex. In other aspects of the present disclosure, a nucleic acid molecule or an expression cassette encoding a CAR or an exogenous TCR is integrated into the genome of the cell or, in one alternative embodiment, is not integrated into the genome of the cell.

In some embodiments, the nucleic acid encoding a CAR or an exogenous TCR is introduced into a cell using any technology known in the art. In specific embodiments, vectors or expression cassettes comprising a nucleic acid encoding a CAR or an exogenous TCR is introduced into a cell using a viral vector (i.e., a virus). Such vectors are known in the art and include lentiviruses (i.e., lentiviral vectors), adenoviruses (i.e., adenoviral vectors), and adeno-associated viruses (i.e., AAV vectors) (reviewed in Vannucci, et al. (2013 New Microbiol. 36: 1-22). Recombinant AAVs useful in the present disclosure can have any serotype suitable for transduction of the virus into the cell and insertion of a nuclease gene into the cell and, in particular embodiments, into the cell genome. In particular embodiments, recombinant AAVs have a serotype of AAV2, AAV6, or AAV8. Recombinant AAVs can also be self-complementary such that they do not require second-strand DNA synthesis in the host cell (McCarty, et al. (2001) Gene Ther. 8: 1248-54).

In some embodiments, nucleic acid molecules disclosed herein are delivered into a cell in the form of DNA (e.g., circular or linearized plasmid DNA or PCR products) or RNA. In some embodiments, wherein engineered nuclease genes are delivered in DNA form (e.g. plasmid) and/or via a virus (e.g. AAV or lentivirus), the genes are operably linked to a promoter or found on an expression cassette, as described herein. In some embodiments, the promoter is a viral promoter, such as an endogenous promoter from a viral vector (e.g. the LTR of a lentiviral vector) or the well-known cytomegalovirus- or SV40 virus-early promoters. In other embodiments, the promoter is a synthetic promoter, such as the JeT promoter. In certain embodiments, genes encoding a CAR or an exogenous TCR are operably linked to a promoter (or promoters) that drives gene expression preferentially in the target cell (e.g., a human T cell or a human NK cell).

In some embodiments, nucleic acid molecules encoding a CAR or an exogenous TCR are coupled covalently or non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nucleic acid molecules or expression cassettes can be attached to or encapsulated with the nanoparticle core. This increases the copy number of the DNA that is delivered to each cell and, so, increases the intracellular expression of each nucleic acid molecule to maximize the likelihood that a CAR or an exogenous TCR will be expressed in the cell. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, nucleic acid molecules encoding a CAR or an exogenous TCR are encapsulated within liposomes or complexed using cationic lipids (see, e.g., Lipofectamine, Life Technologies Corp., Carlsbad, Calif.; Zuris et al. (2015) NatBiotechnol. 33: 73-80; Mishra et al. (2011) J Drug Deliv. 2011:863734). The liposome and lipoplex formulations can protect the payload from degradation, and facilitate cellular uptake and delivery efficiency through fusion with and/or disruption of the cellular membranes of the cells.

In some embodiments, nucleic acid molecules encoding a CAR or an exogenous TCR are encapsulated within polymeric scaffolds (e.g., PLGA) or complexed using cationic polymers (e.g., PEI, PLL) (Tamboli et al. (2011) Ther Deliv. 2(4): 523-536). In some embodiments, nucleic acid molecules or expression cassettes encoding a c CAR or an exogenous TCR are combined with amphiphilic molecules that self-assemble into micelles (Tong et al. (2007) Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions outside of the cell.

In some embodiments, nucleic acid molecules encoding a CAR or an exogenous TCR are formulated as emulsions for delivery to the cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in US Patent Application Nos. 2002/0045667 and 2004/0043041, and U.S. Pat. Nos. 6,015,832, 6,506,803, 6,635,676, and 6,559,189, each of which is incorporated herein by reference in its entirety.

In some embodiments, nucleic acid molecules encoding a CAR or an exogenous TCR are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) Pharm Sci. 97(1): 123-43). The dendrimer generation can control the payload capacity and size, and can provide a high payload capacity. Moreover, display of multiple surface groups can be leveraged to improve stability and reduce nonspecific interactions.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, the invention further provides for the introduction of the nucleic acid molecules disclosed herein into a target gene to produce a cell having no detectable cell surface expression of an endogenous TCR, or a component thereof (e.g., CD3). In certain embodiments, the nucleic molecules are introduced into a recognition sequence present in the target gene, which comprises the coding sequences for a polypeptide.

For example, in some embodiments, the invention provides for the introduction of the nucleic acid molecules disclosed herein into the T cell receptor alpha gene to produce a cell having no detectable cell surface expression of an endogenous TCR, or a component thereof (e.g., CD3). In certain embodiments, the nucleic molecules are introduced into a recognition sequence present in the T cell receptor alpha constant region gene, which comprises the coding sequences for the T cell receptor alpha subunit. As such, introduction of the nucleic acid molecules disrupts expression of the endogenous T cell receptor alpha subunit, and consequently disrupts expression of the endogenous T cell receptor at the cell surface. Without the endogenous T cell receptor, cells will also lack CD3 on the cell surface. In particular embodiments, such recognition sequences can be present within exon 1 of the T cell receptor alpha constant region gene.

In some embodiments, the invention further provides for the introduction of the nucleic acid molecules disclosed herein into the T cell receptor beta gene to produce a cell having no detectable cell surface expression of an endogenous TCR, or a component thereof (e.g., CD3). In certain embodiments, the nucleic molecules are introduced into a recognition sequence present in the T cell receptor beta constant region gene, which comprises the coding sequences for the T cell receptor beta subunit. As such, introduction of the nucleic acid molecules disrupts expression of the endogenous T cell receptor alpha subunit, and consequently prevents expression of the endogenous T cell receptor at the cell surface.

In some embodiments, the invention further provides for the introduction of an inhibitory nucleic acid molecule in a genetically modified cell according to the invention, which targets a gene found in T cells (e.g., a component of the endogenous TCR and/or CD3). For example, the inhibitory nucleic acid molecule may target the T cell receptor alpha subunit or beta subunit and prevent production of the T cell receptor alpha subunit or beta subunit peptides thereby limiting expression of CD3 on the cell surface. Alternatively, the inhibitory nucleic acid may target CD3 directly or target both a component of the endogenous TCR and CD3. It is contemplated herein that 1, 2, 3, 4 or more inhibitory nucleic acids may be used to reduce expression of a gene found in a T cell (e.g., a component of the endogenous TCR and/or CD3). Exemplary and non-limiting inhibitory nucleic acid molecules include, without limitation, an RNA interference molecule such as a short hairpin RNA (shRNA), a small interfering RNA (siRNA), a hairpin siRNA, a microRNA (miRNA), or a precursor miRNA. Inhibitory nucleic acid molecules can further include microRNA-adapted shRNAs.

The use of nucleases for disrupting expression of an endogenous TCR gene has been disclosed, including the use of zinc finger nucleases (ZFNs), transcription activator-like effector nucleases (TALENs), megaTALs, and CRISPR systems (e.g., Osborn et al. (2016), Molecular Therapy 24(3): 570-581; Eyquem et al. (2017), Nature 543: 113-117; U.S. Pat. No. 8,956,828; U.S. Publication No. US2014/0301990; U.S. Publication No. US2012/0321667). The specific use of engineered meganucleases for cleaving DNA targets in the human TRAC gene has also been previously disclosed. For example, International Publication No. WO 2014/191527, which disclosed variants of the I-OnuI meganuclease that were engineered to target a recognition sequence within exon 1 of the TCR alpha constant region gene. Moreover, in International Publication Nos. WO 2017/062439 and WO 2017/062451, Applicants disclosed engineered meganucleases which have specificity for recognition sequences in exon 1 of the TCR alpha constant region gene. These included “TRC 1-2 meganucleases” which have specificity for the TRC 1-2 recognition sequence (SEQ ID NO: 1) in exon 1 of the TRAC gene. The '439 and '451 publications also disclosed methods for targeted insertion of a CAR coding sequence or an exogenous TCR coding sequence into a cleavage site in the TCR alpha constant region gene.

Any engineered nuclease can be used for targeted insertion of the donor template, including an engineered meganuclease, a zinc finger nuclease, a TALEN, a compact TALEN, a CRISPR system nuclease, or a megaTAL.

For example, zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). A CRISPR system comprises two components: (1) a CRISPR nuclease; and (2) a short “guide RNA” comprising a ˜20 nucleotide targeting sequence that directs the nuclease to a location of interest in the genome. The CRISPR system may also comprise a tracrRNA. By expressing multiple guide RNAs in the same cell, each having a different targeting sequence, it is possible to target DNA breaks simultaneously to multiple sites in the genome.

Engineered meganucleases that bind double-stranded DNA at a recognition sequence that is greater than 12 base pairs can be used for the presently disclosed methods. A meganuclease can be an endonuclease that is derived from I-CreI and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker.

Nucleases referred to as megaTALs are single-chain endonucleases comprising a transcription activator-like effector (TALE) DNA binding domain with an engineered, sequence-specific homing endonuclease.

A transgene described herein can be inserted at any position within, for example, the TCR alpha gene, the TRAC gene, the TCR beta gene, or the TRBC gene, such that insertion of the transgene results in disrupted expression of the endogenous polypeptide; i.e., the endogenous TCR alpha chain or the endogenous TCR beta chain. In some examples, the transgene can be inserted in the TRAC gene at a meganuclease recognition sequence comprising SEQ ID NO: 1. In particular examples, the transgene is inserted between positions 13 and 14 of SEQ ID NO: 1.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. For example, nuclease-mediated insertion using engineered single-chain meganucleases has been disclosed in International Publication Nos. WO 2017/062439 and WO 2017/062451. Nuclease-mediated insertion of the donor template can also be accomplished using an engineered single-chain meganuclease comprising SEQ ID NO: 12.

In some embodiments, mRNA encoding the engineered nuclease is delivered to the cell because this reduces the likelihood that the gene encoding the engineered nuclease will integrate into the genome of the cell.

The mRNA encoding an engineered nuclease can be produced using methods known in the art such as in vitro transcription. In some embodiments, the mRNA comprises a modified 5′ cap. Such modified 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, Calif.), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element. The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In particular embodiments, nucleoside modifications can include a modification of uridine to pseudouridine, and/or a modification of uridine to N1-methyl pseudouridine.

In another particular embodiment, a nucleic acid encoding an engineered nuclease can be introduced into the cell using a single-stranded DNA template. The single-stranded DNA can further comprise a 5′ and/or a 3′ AAV inverted terminal repeat (ITR) upstream and/or downstream of the sequence encoding the engineered nuclease. In other embodiments, the single-stranded DNA can further comprise a 5′ and/or a 3′ homology arm upstream and/or downstream of the sequence encoding the engineered nuclease.

In other embodiments, genes encoding a nuclease of the invention are introduced into a cell using a linearized DNA template. Such linearized DNA templates can be produced by methods known in the art. For example, a plasmid DNA encoding a nuclease can be digested by one or more restriction enzymes such that the circular plasmid DNA is linearized prior to being introduced into a cell.

Purified engineered nuclease proteins, or nucleic acids encoding engineered nucleases, can be delivered into cells to cleave genomic DNA by a variety of different mechanisms known in the art, including those detailed herein for introducing transgenes into a cell.

### 2.11 Methods of Administering Genetically-Modified Cells

Another aspect disclosed herein is the administration of the genetically-modified cells of the present disclosure (e.g., T cells modified to express a CAR or an exogenous TCR and do not express CD3 on the cell surface) to a subject in need thereof. For example, an effective amount of a population of genetically-modified cells can be administered to a subject having a disease, symptoms of a disease, or markers of a disease. In particular embodiments, the disease can be cancer, and administration of the genetically-modified immune cells of the invention represent an immunotherapy, such as an allogeneic cellular immunotherapy.

For example, an effective amount of a population of cells comprising an exogenous polynucleotide described herein, which express a cell-surface CAR or an exogenous TCR, can be administered to a subject having a disease. Thus, the present disclosure also provides a method for providing a T cell-mediated immune response to a target cell population or tissue in a mammal, comprising the step of administering to the mammal a CAR T cell, wherein the CAR comprises an extracellular ligand-binding domain that specifically interacts with a predetermined target, such as a tumor antigen, and an intracellular domain that comprises at least one signaling domain, such as CD3ζ, and optionally one or more co-stimulatory signaling domains. The administered CAR T cells are able to reduce the proliferation, reduce the number, or kill target cells in the recipient. Such methods can also include, for example, the administration of genetically-modified T cell expressing an exogenous TCR, or a genetically-modified NK cell expressing a CAR or an exogenous TCR. Unlike antibody therapies, genetically-modified cells of the present disclosure are able to replicate and expand in vivo, resulting in long-term persistence that can lead to sustained control of a disease.

In certain embodiments, a population of genetically-modified cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified cell described herein (e.g., a population of cells modified to express a chimeric antigen receptor (CAR) or an exogenous T cell receptor (TCR) and do not express CD3 on the cell surface).

Additionally, to promote expansion of the genetically-modified cells and reduce the likelihood or severity of host vs graft rejection, the subject may undergo a lymphodepletion regimen described herein prior to, concomitant with, or following administration of the genetically-modified cells. In particular embodiments, the subject is administered a lymphodepletion therapy comprising a lymphodepleting chemotherapeutic agent and/or an antibody or antigen-binding fragment thereof that specifically binds CD3 (i.e., an anti-CD3 antibody or antigen-binding fragment thereof), as provided herein. The subject may be administered the genetically-modified cells described herein (e.g., cells modified to express a chimeric antigen receptor (CAR) or an exogenous T cell receptor (TCR) and do not express CD3 on the cell surface), such as from the same physician that performed the lymphodepletion therapy or from a different physician.

The subject may be administered the genetically-modified cells, for instance, at a dosage of from 1×10³ to 1×10⁹ genetically-modified cells/kg. Thus, in some embodiments, the subject is administered genetically-modified cells described herein at a dosage of about 1×10³ to about 1×10⁹ genetically-modified cells/kg. In some embodiments, the subject is administered genetically-modified cells described herein at a dosage of about 1×10⁴ to about 1×10⁷ genetically-modified cells/kg. In some embodiments, the subject is administered genetically-modified cells described herein at a dosage of about 1×10⁵ to about 1×10⁶ genetically-modified cells/kg.

Examples of possible routes of administration of compositions comprising genetically-modified cells or lymphodepletion regimens described herein include parenteral, (e.g., intravenous (IV), intramuscular (IM), intradermal, subcutaneous (SC), or infusion) administration. Moreover, the administration may be by continuous infusion or by single or multiple boluses. In specific embodiments, one or both of the agents is infused over a period of less than about 12 hours, less than about 10 hours, less than about 8 hours, less than about 6 hours, less than about 4 hours, less than about 3 hours, less than about 2 hours, or less than about 1 hour. In still other embodiments, the infusion occurs slowly at first and then is increased over time.

In some embodiments, a genetically-modified cell of the present disclosure targets a tumor antigen for the purposes of treating cancer. Such cancers can include, without limitation, those cancers described elsewhere herein.

In some of these embodiments wherein cancer is treated with the presently disclosed genetically-modified cells, the subject administered the genetically-modified cells is further administered an additional therapeutic agent or treatment, including, but not limited to gene therapy, radiation, surgery, or a chemotherapeutic agent(s) (i.e., chemotherapy).

When an “effective amount” or “therapeutic amount” is indicated, the precise amount of the compositions of the present disclosure to be administered can be determined by a physician with consideration of individual differences in age, weight, tumor size (if present), extent of infection or metastasis, and condition of the subject. In some embodiments, a pharmaceutical composition comprising the genetically-modified cells described herein is administered at a dosage of 10³ to 10⁹ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The genetically-modified cells can be administered by using infusion techniques that are commonly known in immunotherapy (see, e.g., Rosenberg et al., New Eng. J. of Med. 319: 1676, 1988). The optimal dosage and treatment regimen for a particular subject can readily be determined by one skilled in the art of medicine by monitoring the subject for signs of disease and adjusting the treatment accordingly.

In some embodiments, the administration of genetically-modified cells of the present disclosure reduces at least one symptom of a target disease or condition. For example, administration of genetically-modified cells of the present disclosure can reduce at least one symptom of a cancer, such as cancers of B-cell origin. Symptoms of cancers, such as cancers of B-cell origin, are well known in the art and can be determined by known techniques.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Use of Anti-CD3 Antibodies in Preventing CAR T Cell Lysis

### Methods

A leukopheresis product was sourced from HemaCare (from subject D270185: a healthy, consented donor). T cells were enriched using CliniMACS anti-human CD4 and CD8 microbeads and a CliniMACS separator (Miltenyi Biotec). T cells were stimulated with TransAct anti-CD3/CD28 (Miltenyi Biotec) for three days in Xuri T cell expansion medium (GE Life Sciences) supplemented with 5% pooled human AB serum (Innovative Research) and 10 ng/ml interleukin-2 (IL-2: CellGenix). On day 3, T cells received lpg of an mRNA (TriLink) encoding the TRC 1-2L.1592 meganuclease (see PCT international patent application no. PCT/US2019/027019) per 1×10⁶ cells via electroporation using a Lonza 4-D Nucleofector. Electroporated cells were immediately transduced with AAV7206 at an MOI of 20,000 viral genomes per cell. Five days following electroporation/transduction CD3+ cells were depleted using a CD3 selection kit from StemCell Technologies.

To measure susceptibility to a CD3 depleting antibody, a complement-dependent cytotoxicity (CDC) assay was conducted. 2.0×10⁵ cells were plated in wells of a round-bottom 96 well plate in 200 μl of Xuri medium supplemented with 30% off-the-clot human serum (Innovative Research) and the indicated concentrations of ATGAM (equine anti-human thymocyte globulin, Pfizer) or a foralumab biosimilar (Creative Biolabs). Cultures were incubated for 24 h at 37° C. prior to labeling with 1 μg/ml propidium iodide (Sigma) and analyzing for live cell number using a CytoFLEX-LX (Beckman-Coulter).

### Results

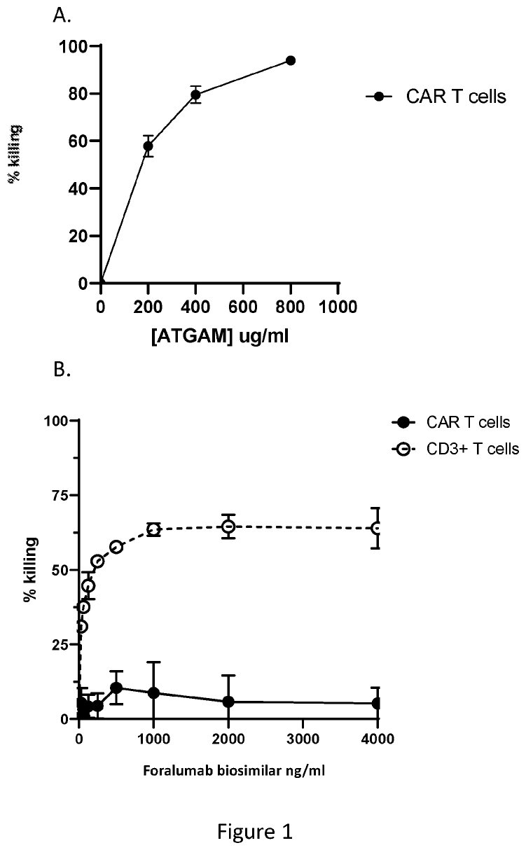

CD3-depleted CAR T cell preparations were exposed to ATGAM and percent killing was calculated (FIG. 1A). Relative to a no Ab control, approximately all CAR T cells were killed in the presence of 800 μg/ml of ATGAM. Approximately 80% were killed in the presence of 400 μg/ml and approximately half were killed in the presence of 200 μg/ml. This indicates that serum activity and assay time were appropriate to visualize a range of antibody-mediated cytotoxicity efficiencies.

CD3-depleted CAR T preparations, or non-edited CD3⁺ T cells were exposed to the indicated concentrations of a foralumab biosimilar (30-4000 ng/ml) and percent cytolysis appears in FIG. 1B. Relative to zero antibody control samples, only modest levels of CDC are observed in CAR T samples (10% or less) and CDC was not found to increase with a foralumab biosimilar concentration. CD3⁺ control T cells, however, are effectively killed at all concentrations tested, with dose-dependent increases evident when the foralumab biosimilar was present at less than 1000 ng/ml and maximal CDC levels of approximately 65% killing.

### CONCLUSIONS

The foralumab biosimilar effectively mediates the complement-dependent lysis of CD3⁺ T cells, but TRAC-edited CAR T cells are resistant to this drug. It is expected that TRAC-edited CAR T cells in a patient that receives a foralumab biosimilar after CAR T infusion will selectively survive depletion while the patient's endogenous T cells are eliminated, perhaps delaying the rejection of CAR T cells by the host's immune system.

1. A method of immunotherapy for treating a cancer in a subject in need thereof, said method comprising:
(a) administering to said subject an antibody, or antigen-binding fragment thereof, that specifically binds CD3 in an amount effective to deplete a population of lymphocytes in said subject; and
(b) administering to said subject a composition comprising a population of genetically-modified T cells that have no detectable CD3 on the cell surface,
wherein said population of genetically-modified T cells comprise in their genome an exogenous polynucleotide encoding a chimeric antigen receptor (CAR) or an exogenous T cell receptor (TCR) that is expressed by said genetically-modified T cells.

2. The method of claim 1, wherein said method further comprises administering a lymphodepleting chemotherapeutic agent or an additional lymphodepleting antibody to said subject prior to administration of said composition comprising said population of genetically-modified T cells.

3. The method of claim 1 or claim 2, wherein said antibody, or antigen binding fragment thereof, is administered to said subject prior to administration of said composition comprising said population of genetically-modified T cells.

4. The method of claim 1 or claim 2, wherein said antibody, or antigen binding fragment thereof, is administered to said subject concomitant with administration of said composition comprising said population of genetically-modified T cells.

5. The method of claim 1 or claim 2, wherein said antibody, or antigen binding fragment thereof, is administered to said subject following administration of said composition comprising said population of genetically-modified T cells.

6. A method of immunotherapy for treating a cancer in a subject in need thereof, said method comprising administering to said subject a composition comprising a population of genetically-modified T cells that have no detectable CD3 on the cell surface,
wherein said population of genetically-modified T cells comprise in their genome an exogenous polynucleotide encoding a chimeric antigen receptor (CAR) or an exogenous T cell receptor (TCR) that is expressed by said genetically-modified T cells; and
wherein said subject has previously been administered a lymphodepleting chemotherapeutic agent and an antibody or antigen-binding fragment thereof that specifically binds CD3 in an amount effective to deplete a population of lymphocytes in said subject.

7. A method of immunotherapy for treating a cancer in a subject in need thereof, said method comprising administering to said subject an antibody, or antigen-binding fragment thereof, that specifically binds CD3 in an amount effective to deplete a population of lymphocytes in said subject;
wherein said subject has previously been administered a composition comprising a population of genetically-modified T cells that have no detectable CD3 on the cell surface,
wherein said population of genetically-modified T cells comprise in their genome an exogenous polynucleotide encoding a chimeric antigen receptor (CAR) or an exogenous T cell receptor (TCR) that is expressed by said genetically-modified T cells.

8. The method of any one of claims 1-7, wherein said antibody, or antigen binding fragment thereof, is administered to said subject 1-30 days prior to administration of said population of genetically-modified T cells.

9. The method of any one of claims 2-6, wherein said antibody, or antigen binding fragment thereof, is administered to said subject prior to administration of said lymphodepleting chemotherapeutic agent.

10. The method of any one of claims 2-6, wherein said antibody, or antigen binding fragment thereof, is administered to said subject concomitant with administration of said lymphodepleting chemotherapeutic agent.

11. The method of any one of claims 2-6, wherein said antibody, or antigen binding fragment thereof, is administered to said subject following administration of said lymphodepleting chemotherapeutic agent.

12. The method of any one of claims 1-11, wherein said antibody, or antigen binding fragment thereof, is administered to said subject at a dose of from about 0.01 mg/kg to about 1.0 mg/kg.

13. The method of any one of claims 1-12, wherein said antibody, or antigen-binding fragment thereof, is selected from the group consisting of a monoclonal antibody, a polyclonal antibody, a humanized antibody, a fully human antibody, a bispecific antibody, a dual-variable immunoglobulin domain, a single-chain Fv molecule (scFv), a sdAb, a diabody, a triabody, a nanobody, an antibody-like protein scaffold, a Fv fragment, a Fab fragment, a F(ab′)₂ molecule, and a tandem di-scFv.

14. The method of any one of claims 1-13, wherein said antibody, or antigen-binding fragment thereof, does not detectably bind said genetically-modified T cells.

15. The method of any one of claims 1-14, wherein said composition comprising said population of genetically-modified T cells is administered to said subject at a dose of 1×10³ to 1×10⁹ genetically-modified cells/kg.

16. The method of any one of claims 2-15, wherein said lymphodepleting chemotherapeutic agent is administered three or more days prior to administration of said composition comprising said population of genetically-modified T cells.

17. The method of any one of claims 2-15, wherein said lymphodepleting chemotherapeutic agent is administered seven days or less prior to administration of said composition comprising said population of genetically-modified T cells.

18. The method of any one of claims 2-17, wherein said lymphodepleting chemotherapeutic agent is fludarabine, cyclophosphamide, bendamustine, melphalan, 6-mercaptopurine (6-MP), daunorubicin, cytarabine, L-asparaginase, methotrexate, prednisone, dexamethasone, nelarabine, and said additional lymphodepleting antibody is an anti-CD52 antibody (e.g., alemtuzumab) or rituximab, or a combination thereof.

19. The method of claim 18, wherein cyclophosphamide is administered to said subject at a dose of about 250-1500 mg/m²/day.

20. The method of claim 18, wherein cyclophosphamide is administered to said subject at a dose of about 500-1000 mg/m²/day.

21. The method of claim 18, wherein cyclophosphamide is administered to said subject at a dose of about 500 mg/m²/day.

22. The method of claim 21, wherein cyclophosphamide is administered to said subject at a dose of about 500 mg/m²/day daily starting five days and ending two to three days prior to administration of said composition comprising said population of genetically-modified T cells.

23. The method of claim 18, wherein cyclophosphamide is administered to said subject at a dose of about 1000 mg/m²/day.

24. The method of claim 23, wherein cyclophosphamide is administered to said subject at a dose of about 1000 mg/m²/day daily starting four days and ending two to three days prior to administration of said composition comprising said population of genetically-modified T cells.

25. The method of claim 18, wherein fludarabine is administered to said subject at a dose of 10-40 mg/m²/day.

26. The method of claim 18, wherein fludarabine is administered to said subject at a dose of 30 mg/m²/day.

27. The method of claim 26, wherein fludarabine is administered to said subject at a dose of about 30 mg/m²/day daily starting five days and ending two to three days prior to administration of said composition comprising said population of genetically-modified T cells.

28. The method of claim 26, wherein fludarabine is administered to said subject at a dose of about 30 mg/m²/day daily starting seven days and ending two to three days prior to administration of said composition comprising said population of genetically-modified T cells.

29. The method of any one of claims 2-28, wherein said lymphodepleting chemotherapeutic agent is administered in combination with an additional cancer therapy selected from the group consisting of an additional chemotherapeutic agent, surgery, radiation, and gene therapy.

30. The method of any one of claims 1-29, wherein said CAR or said exogenous TCR specifically binds to a molecule on the surface of a cancer cell.

31. The method of claim 30, wherein said CAR specifically binds to CD19, CD20, BCMA, CLL1, CS1 (SLAMF7), MUC1, FLT3, HPV16 E6, or HPV16 E7.

32. The method of any one of claims 1-31, wherein said exogenous polynucleotide is within a target gene in the genome of said genetically-modified T cell.

33. The method of claim 32, wherein said target gene is selected from the group consisting of a TCR alpha gene, a TCR alpha constant (TRAC) gene, a TCR beta gene, or a TCR beta constant (TRBC) gene.

34. The method of any one of claims 1-33, wherein said genetically-modified T cells have no detectable cell surface expression of an endogenous T cell receptor.

35. The method of any one of claims 1-34, wherein said genetically-modified T cell is a human T cell, or a cell derived therefrom.

36. The method of any one of claims 1-35, wherein said cancer is selected from the group consisting of a cancer of carcinoma, lymphoma, sarcoma, blastomas, myeloma, and leukemia.

37. The method of any one of claims 1-36, wherein said cancer is selected from the group consisting of lung cancer, melanoma, breast cancer, prostate cancer, colon cancer, renal cell carcinoma, ovarian cancer, neuroblastoma, rhabdomyosarcoma, leukemia, lymphoma, acute lymphoblastic leukemia, multiple myeloma, small cell lung cancer, Hodgkin's lymphoma, and childhood acute lymphoblastic leukemia.

38. The method of any one of claims 1-35, wherein said cancer is selected from the group consisting of a cancer of B-cell origin.

39. The method of claim 38, wherein said cancer of B-cell origin is selected from the group consisting of B-lineage acute lymphoblastic leukemia, B-cell chronic lymphocytic leukemia, and B-cell non-Hodgkin's lymphoma.

40. A kit comprising:
(a) an antibody, or antigen-binding fragment thereof, that specifically binds CD3; and
(b) a composition comprising a population of genetically-modified T cells,
wherein said population of genetically-modified T cells comprise in their genome an exogenous polynucleotide encoding a chimeric antigen receptor (CAR) or an exogenous T cell receptor (TCR) that is expressed by said genetically-modified T cells.

41. A kit comprising:
(a) a lymphodepleting chemotherapeutic agent,
(b) an antibody, or antigen-binding fragment thereof, that specifically binds CD3; and
(c) a composition comprising a population of genetically-modified T cells, wherein said population of genetically-modified T cells comprise in their genome an exogenous polynucleotide encoding a chimeric antigen receptor (CAR) or an exogenous T cell receptor (TCR) that is expressed by said genetically-modified T cells.

42. The kit of claim 41, wherein said lymphodepleting chemotherapeutic agent is fludarabine, cyclophosphamide, or a combination thereof.

43. The kit of any one of claims 40-42, wherein said exogenous polynucleotide is within a target gene in the genome of said genetically-modified T cell.

44. The kit of claim 43, wherein said target gene is selected from the group consisting of TCR alpha gene, a TRAC gene, a TCR beta gene, or a TRBC gene.

45. The kit of any one of claims 40-44, wherein said genetically-modified T cell is a human T cell, or a cell derived therefrom.

46. The kit of any one of claims 40-45, wherein said CAR or said exogenous TCR specifically binds to a molecule on the surface of a cancer cell.

47. The kit of claim 46, wherein said CAR specifically binds to CD19, CD20, BCMA, or CLL1.

48. The kit of claim any one of claims 40-47, wherein said kit further comprises instructions for use of components of the kit in treating a cancer.
